(19) United States
(12) Patent Application Publication
(10) Pub. No.: US 2026/0130795 A1
(43) Pub. Date: May 14, 2026

(54) SYSTEMS AND METHODS FOR DRUG DELIVERY TO OCULAR TISSUE

(71) Applicant: Regeneron Pharmaceuticals, Inc., Tarrytown, NY (US)

(72) Inventors: Bryan C. GRYGUS, Clifton Park, NY (US); Trevor LANGLEY, Rensselaer, NY (US); Angel ZAREV, Newmarket (GB); Fraser MCKAY, Cambridge (GB); Matthew BARTY, Cambridge (GB); Ted PEACHEY, Melbourne (GB)

(73) Assignee: Regeneron Pharmaceuticals, Inc., Tarrytown, NY (US)

(21) Appl. No.: 19/385,975

(22) Filed: Nov. 11, 2025

Related U.S. Application Data
(60) Provisional application No. 63/719,162, filed on Nov. 12, 2024.

(51) Int. Cl. A61F 9/00

(52) U.S. Cl. CPC A61F 9/0026; A61F 9/0017; A61F 2250/0097

(57) ABSTRACT

Disclosed are devices and methods for directed delivery of a medicament into ocular tissue. A device for delivering a medicament to ocular tissue may include a housing, a container configured to contain the medicament, and a needle in fluid communication with the container, and a carriage positioned within the housing and coupled to the container and the needle. The carriage may be translatable along the needle axis and the carriage may include a threaded outer surface. The device may further include a nut positioned within the housing and engaged with the threaded outer surface. Rotation of the nut may be configured to cause the carriage and the needle to translate in a distal direction along the needle axis.

### CROSS-REFERENCE TO RELATED APPLICATION(S)

This application claims priority to U.S. Provisional Patent Application No. 63/719,162, filed on Nov. 12, 2024, the entirety of which is incorporated herein by reference.

### TECHNICAL FIELD

Various aspects of the present disclosure relate generally to delivering drugs to ocular tissue. Certain aspects of the present disclosure relate to instruments and related methods for delivering drugs to, e.g., the suprachoroidal space of an eye.

### INTRODUCTION

Eye conditions and diseases lead to optic nerve damage and visual field loss. Medications, laser surgery, and/or incisional surgery may be employed to help lower intraocular pressure, preserve a subject's existing vision, and delay further progression of a condition and/or disease. Devices for delivering drugs to ocular tissue, and methods of using such devices, are sought after to provide improved outcomes for users and subjects. It is particularly desirable for such devices to facilitate accurate delivery of medicament to target regions of an eye, cause minimal damage to the ocular tissue during use, and/or minimize discomfort to a subject receiving drug therapy.

### SUMMARY OF THE DISCLOSURE

According to an aspect of the disclosure, a device for delivering a medicament to ocular tissue may include: a housing; a container configured to contain the medicament; and a needle in fluid communication with the container. The needle defines a needle axis. The device may further include a carriage positioned within the housing and coupled to the container and the needle. The carriage may be translatable along the needle axis, and may include a threaded outer surface. The device may further include a nut positioned within the housing and engaged with the threaded outer surface. Rotation of the nut may be configured to cause the carriage and the needle to translate in a distal direction along the needle axis.

According to another aspect of the disclosure, a device for delivering a medicament to ocular tissue may include: a housing; a needle defining a needle axis; and a carriage positioned within the housing and coupled to the needle. The carriage may be translatable along the needle axis and may include a threaded outer surface. The device may further include a nut positioned within the housing and engaged with the threaded outer surface. The device may further include a motor coupled to the nut and a controller configured to: receive an activation signal; and cause, in response to the activation signal, the motor to apply a torque to the nut, thereby causing the nut to rotate and causing the carriage and the needle to translate distally along the needle axis.

In still another aspect of the disclosure, a device for delivering a medicament to ocular tissue may include a container configured to contain the medicament and a needle in fluid communication with the container. The needle may define a needle axis. The device may further include a collar coupled to the container and the needle. The collar may include a threaded outer surface. The device may further include an eye shield coupled to a distal end of the device. The eye shield may include a contoured surface configured to conform to an outer surface of an eye and the eye shield may be configured to translate along the needle axis relative to the collar. The device may further include a wheel engaged with the threaded outer surface. Rotation of the wheel may be configured to cause the eye shield to translate along the needle axis

### BRIEF DESCRIPTION OF THE FIGURES

The accompanying drawings, which are incorporated in and constitute a part of this specification, illustrate various examples and, together with the description, serve to explain the principles of the disclosed examples and embodiments.

Aspects of the disclosure may be implemented in connection with embodiments illustrated in the attached drawings. These drawings show different aspects of the present disclosure and, where appropriate, reference numerals illustrating like structures, components, materials, and/or elements in different figures are labeled similarly. It is understood that various combinations of the structures, components, and/or elements, other than those specifically shown, are contemplated and are within the scope of the present disclosure.

Moreover, there are several embodiments described and illustrated herein. The present disclosure is neither limited to any single aspect or embodiment, nor is it limited to any combinations and/or permutations of such aspects and/or embodiments. Moreover, each of the aspects of the present disclosure, and/or embodiments thereof, may be employed alone or in combination with one or more of the other aspects of the present disclosure and/or embodiments thereof. For the sake of brevity, certain permutations and combinations are not discussed and/or illustrated separately herein. Notably, an embodiment or implementation described herein as “exemplary” is not to be construed as preferred or advantageous, for example, over other embodiments or implementations; rather, it is intended to reflect or indicate the embodiment(s) is/are “example” embodiment(s).

FIG. 1A is a perspective view of an exemplary instrument for treating ocular tissue, according to an embodiment of the present disclosure.

FIG. 1B is a front view of the exemplary instrument for treating ocular tissue of FIG. 1A.

FIG. 2A is a cross-sectional view of a needle assembly of an exemplary instrument for treating ocular tissue, according to an embodiment of the present disclosure.

FIG. 2B is an exploded view of the needle assembly of FIG. 2A.

FIG. 3A is an optical coherence tomography image of optical tissue, according to an embodiment of the present disclosure.

FIG. 3B is an optical coherence tomography image of optical tissue, according to an embodiment of the present disclosure.

FIG. 4 depicts an analysis of optical tissue, according to an embodiment of the present disclosure.

FIG. 5A is a side view of an exemplary instrument for treating ocular tissue, according to an embodiment of the present disclosure.

FIG. 5B is a side view of an exemplary instrument for treating ocular tissue, according to an embodiment of the present disclosure.

FIG. 5C is a side view of an exemplary instrument for treating ocular tissue, according to an embodiment of the present disclosure.

FIG. 6A is a perspective view of an exemplary instrument for treating ocular tissue, according to an embodiment of the present disclosure.

FIG. 6B is a side view of the exemplary instrument for treating ocular tissue of FIG. 6A.

FIG. 7 is a cross-sectional view of the exemplary instrument for treating ocular tissue of FIG. 6A.

FIG. 8A is a perspective view of an exemplary instrument for treating ocular tissue, according to an embodiment of the present disclosure.

FIG. 8B is a side view of the exemplary instrument for treating ocular tissue of FIG. 8A.

FIG. 9 is a perspective view of an exemplary instrument for treating ocular tissue, according to an embodiment of the present disclosure.

FIG. 10 is a functional block diagram of an exemplary instrument for treating ocular tissue, according to an embodiment of the present disclosure.

FIG. 11 is a cross-sectional view of an exemplary instrument for treating ocular tissue, according to an embodiment of the present disclosure.

FIG. 12A is a bottom view of an exemplary shield of an instrument for treating ocular tissue, according to an embodiment of the present disclosure.

FIG. 12B is a side view of the exemplary shield of FIG. 12A.

FIG. 12C is a bottom perspective view of the exemplary shield of FIG. 12A.

FIG. 12D is a bottom view of an exemplary sensor array, according to an embodiment of the present disclosure.

FIG. 12E depicts an exemplary instrument for treating ocular tissue including a shield, according to an embodiment of the present disclosure.

FIG. 12F is an alternate view of the exemplary instrument of FIG. 12E.

FIG. 12G is an alternate view of the shield of the exemplary instrument of FIG. 12E.

FIG. 12H depicts an exemplary instrument for treating ocular tissue including a shield, according to an embodiment of the present disclosure.

FIG. 12I is an alternate view of the exemplary instrument of FIG. 12H.

FIG. 12J is an alternate view of the shield of the exemplary instrument of FIG. 12H.

FIG. 12K depicts an exemplary instrument for treating ocular tissue including a shield, according to an embodiment of the present disclosure.

FIG. 12L depicts an exemplary instrument for treating ocular tissue including a shield, according to an embodiment of the present disclosure.

FIG. 12M depicts an exemplary geometry of a shield for an instrument for treating ocular tissue, according to an embodiment of the present disclosure.

FIG. 13 depicts an exemplary sensor for an instrument for treating ocular tissue, according to an embodiment of the present disclosure.

FIG. 14 depicts an exemplary sensor for an instrument for treating ocular tissue, according to an embodiment of the present disclosure.

FIGS. 15A-15C depict an exemplary technique for treating ocular tissue, according to an embodiment of the present disclosure.

FIGS. 16A-16C depict an exemplary technique for treating ocular tissue, according to an embodiment of the present disclosure.

FIGS. 17A-17C depict an exemplary technique for treating ocular tissue, according to an embodiment of the present disclosure.

FIG. 18 is a functional block diagram of an instrument for determining scleral thickness, according to an embodiment of the present disclosure.

FIG. 19A depicts an exemplary needle tip for use with an instrument for treating ocular tissue, according to an embodiment of the present disclosure.

FIG. 19B depicts an exemplary needle tip for an instrument for treating ocular tissue, according to an embodiment of the present disclosure.

FIG. 20 depicts a graphical model of operation of an exemplary instrument for treating ocular tissue, according to an embodiment of the present disclosure.

FIG. 21 is a cross-sectional view of an eye, according to an embodiment of the present disclosure.

Notably, for simplicity and clarity of illustration, certain aspects of the figures depict the general structure and/or manner of construction of the various embodiments. Descriptions and details of well-known features and techniques may be omitted to avoid unnecessarily obscuring other features. Elements in the figures are not necessarily drawn to scale; the dimensions of some features may be exaggerated relative to other elements to improve understanding of the example embodiments. For example, one of ordinary skill in the art would appreciate that the side views are not drawn to scale and should not be viewed as representing proportional relationships between different components. The side views are provided to help illustrate the various components of the depicted assembly, and to show their relative positioning to one another.

### DETAILED DESCRIPTION

Reference will now be made in detail to examples of the present disclosure, which are illustrated in the accompanying drawings. Wherever possible, the same reference numbers will be used throughout the drawings to refer to the same or like parts. The term “distal” refers to a portion farthest away from a user when introducing a device into a subject. By contrast, the term “proximal” refers to a portion closest to the user when placing the device into the subject. In the discussion that follows, relative terms such as “about,” “substantially,” “approximately,” etc. are used to indicate a possible variation of ±10% of a value.

As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” In addition, the terms “first,” “second,” and the like, herein do not denote any order, quantity, or importance, but rather are used to distinguish an element or a structure from another. Moreover, the terms “a” and “an” herein do not denote a limitation of quantity, but rather denote the presence of one or more of the referenced items.

Aspects of the disclosure relate to, among other things, instruments and methods for delivering drugs to ocular tissues. Each of the aspects disclosed herein may include one or more of the features described in connection with any of the other disclosed aspects. It may be understood that both the foregoing general description and the following detailed description are exemplary and explanatory only and are not restrictive of any claimed inventions.

While this disclosure describes certain instruments and methods, additional descriptions relevant to the instruments and methods described herein may be found in U.S. application Ser. No. 17/444,897 (published as US 2022/0047420 A1), U.S. application Ser. No. 18/336,148 (published as US 2023/0405238 A1), U.S. application Ser. No. 18/941,766 (published as US 2025/0143922 A1), U.S. application Ser. No. 15/512,130 (issued as U.S. Pat. No. 11,096,822 B2), U.S. application Ser. No. 15/760,717 (issued as U.S. Pat. No. 11,154,420 B2), U.S. application Ser. No. 16/085,083 (published as US 2021/0169689 A1), U.S. application Ser. No. 16/085,106 (issued as U.S. Pat. No. 11,622,884 B2), and U.S. application Ser. No. 16/646,705 (published as US 2020/0261266 A1), the entireties of which are incorporated herein by reference.

The suprachoroidal space (SCS) is a space between the sclera and choroid, which traverses the circumference of the posterior segment of the eye. The SCS is a useful site for drug delivery because drug delivered to the SCS may target the choroid, retinal pigment epithelium, and/or retina with high bioavailability, while maintaining low levels elsewhere in the eye. Under normal physiological conditions, primarily due to intraocular pressure (IOP), the SCS is primarily in a collapsed state. The SCS plays a role in maintaining IOP via uveoscleral outflow, which is an alternative drainage route for the aqueous humor, and is a natural flow path from the front to the back of the eye. Due to its role in maintaining IOP, the SCS has the potential to expand and contract in response to the presence of fluid. The SCS may expand to accommodate different volumes, for example, up to about 3.0 mm, depending on injection volumes. Injecting high volumes of drugs may have adverse effects, for example, elevated IOP, retinal elevation, choroidal hemorrhage away from needle entry, and choroidal edema and potential choroidal detachment; backflow from needle entry; and reflux of fluid which may cause subconjunctival hemorrhage. Additionally, high volumes of fluid may not be injected into the eye until a needle of an injection device has fully penetrated the sclera.

To expand the SCS, e.g., by separating the sclera and choroid mechanically and breaking down fibers holding the sclera and choroid together, instruments may be inserted through the sclera and placed at the correct depth between the sclera and choroid layers, such that optimal volumes of fluids, e.g., drugs or other suitable therapeutic agents, may be injected into the SCS. Drug delivery into the SCS may allow for direct exposure of the drug to the posterior section of the eye to specifically target, e.g., the retina and/or macula. The SCS may also be a useful destination for slow-release formulations such as depot drugs. For example, a depot drug inserted into the SCS may be useful for treating portions in the rear of the eye, such as the retina, retinal pigment epithelium (RPE), choroid, or other portions. From within the SCS, the depot drug may effectively target portions of the rear of the eye without impinging on a visual axis of the eye. Instruments and methods for insertion and injection into the eye may only allow for extension into a certain depth of the ocular layers. For example, under physiological conditions, the sclera layer ranges from about 300 μm to about 1100 μm, the SCS has a thickness of about 35 μm, and the choroid layer ranges from about 50 μm to about 300 μm. Depth of insertion of an instrument for drug delivery into the ocular layers may range from about 0.5 mm to about 1.1 mm. However, such a depth of insertion may penetrate and/or impact additional layers of the ocular tissue, e.g., the choroid, retinal pigment epithelium (RPE), and retina. Penetration of such layers should be minimized as much as possible, such that the desired drug may be directed into the targeted area of the eye via a minimally invasive procedure. For example, injection procedures may be performed as an outpatient procedure. Instruments and methods discussed in the present disclosure address the challenges of SCS drug delivery described above, and may increase the ability of the SCS to hold and diffuse optimal volumes of drugs, for example, approximately 50 μL to approximately 500 μL. The instruments and methods discussed in the present disclosure may also be used for targeted delivery of therapeutic drugs to regions of the eye other than the SCS.

The embodiments described herein may be used in the treatment of a variety of conditions, including ocular conditions. For example, embodiments of the present disclosure may be used in the treatment of refractive errors, macular degeneration, macular edema, cataracts, retinopathy, retinal detachments, glaucoma, amblyopia, strabismus, any other ocular condition, or any other condition suitable for treatment via tissue in the eye.

The description herein and examples are illustrative and are not intended to be restrictive. One of ordinary skill in the art may make numerous modifications and/or changes without departing from the general scope of the invention. For example, and as has been referenced, aspects of above-described embodiments may be used in any suitable combination with each other. Additionally, portions of the above-described embodiments may be removed without departing from the scope of the disclosure. In addition, modifications may be made to adapt a particular situation or aspect to the teachings of the various embodiments without departing from the scope of the disclosure. Many other embodiments will also be apparent to those of skill in the art upon reviewing the above description.

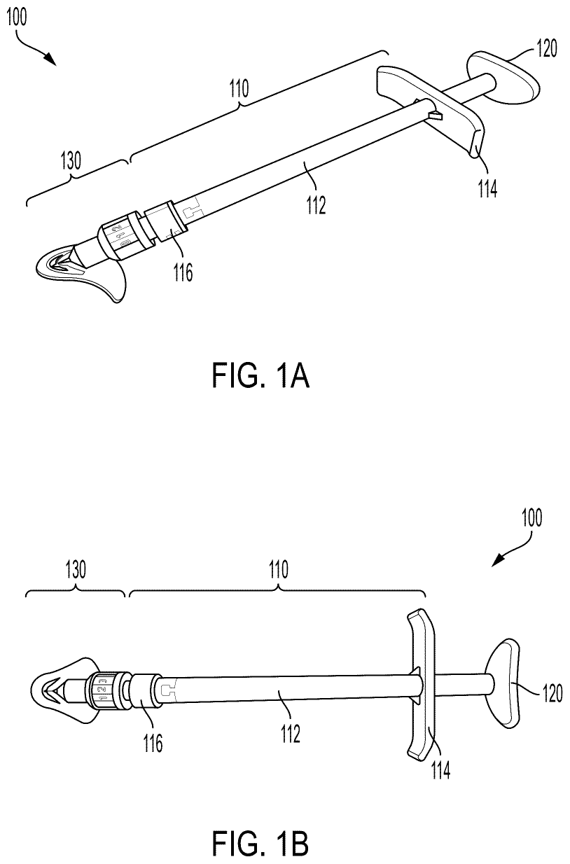

FIGS. 1A and 1B depict an example of an instrument 100 in accordance with the present disclosure. Instrument 100 may include a syringe body 110 having a barrel 112, finger flanges 114, and a receiving portion 116 configured to receive and retain a needle assembly 130. Barrel 112 may be configured to contain a fluid medicament. Instrument 100 may further include a plunger 120. Needle assembly 130 may be configured to be placed against a sclera of a subject's eye. A needle may protrude an adjustable length from needle assembly 130 such that the needle may be controlled to reach a desired depth within a subject's eye. In some embodiments, instrument 100 may be configured such that the needle can be withdrawn into the needle assembly 130. For example, the needle may be withdrawn prior to use to avoid injury.

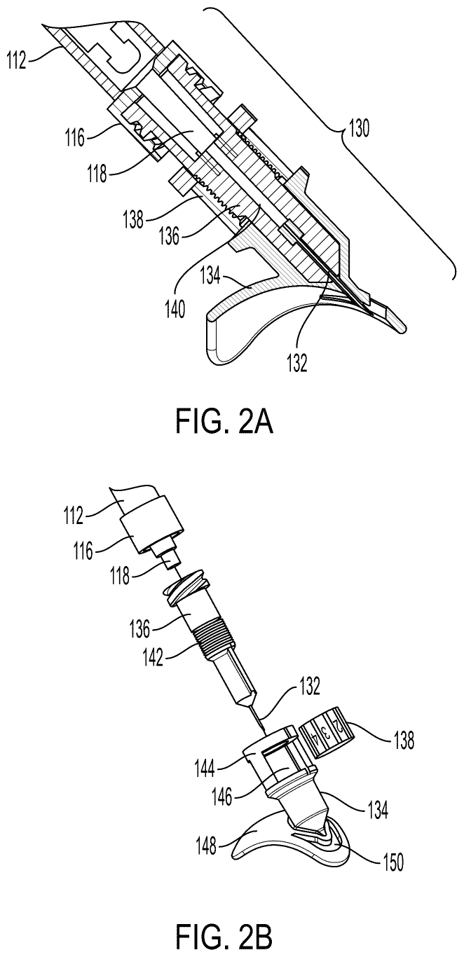

FIGS. 2A and 2B illustrate needle assembly 130 in greater detail. Needle assembly 130 may include a needle 132 having a distal tip, an eye shield 134, a needle collar 136, and an adjustment wheel 138. Needle collar 136 may be coupled to receiving portion 116 of syringe body 110 via a threaded interface, for example. Needle collar 136 may be coupled to receiving portion 116 such that needle 132 is in fluid communication with contents of barrel 112. For example, when instrument 100 is assembled, a fluid medicament within barrel 112 may be expelled through a nozzle 118 of syringe body 110, through a channel 140 of needle collar 136, and through needle 132.

As illustrated in FIG. 2B, needle collar 136 may include a threaded portion 142 to which adjustment wheel 138 may be coupled. Adjustment wheel 138 may nest within eye shield 134 and, in particular, within a chamber 144 of eye shield 134. Eye shield 134 may additionally include a window 146, a flange 148, and a cutout 150. Window 146 may be positioned to allow a user to see and manipulate adjustment wheel 138. For example, adjustment wheel 138 may include markings thereon to indicate to a user a position of adjustment wheel 138 relative to window 146. Flange 148 may provide an increased surface area to be placed in contact with a subject's eye. Flange 148 and other portions of eye shield 134 may be formed of a flexible material in some embodiments to promote comfort. Cutout 150 may allow a user to see a position of needle 132 relative to the subject's eye. Cutout 150 may additionally allow flexion of eye shield 134 to promote comfort.

In some embodiments, portions of eye shield 134 may formed of silicone, rubber, or a combination thereof. In some embodiments, portions of eye shield 134, such as the surface configured to contact a subject's eye, may be textured to inhibit slippage of eye shield 134. For example, portions of eye shield 134 may include protrusions, ridges, or other anti-slip surface features.

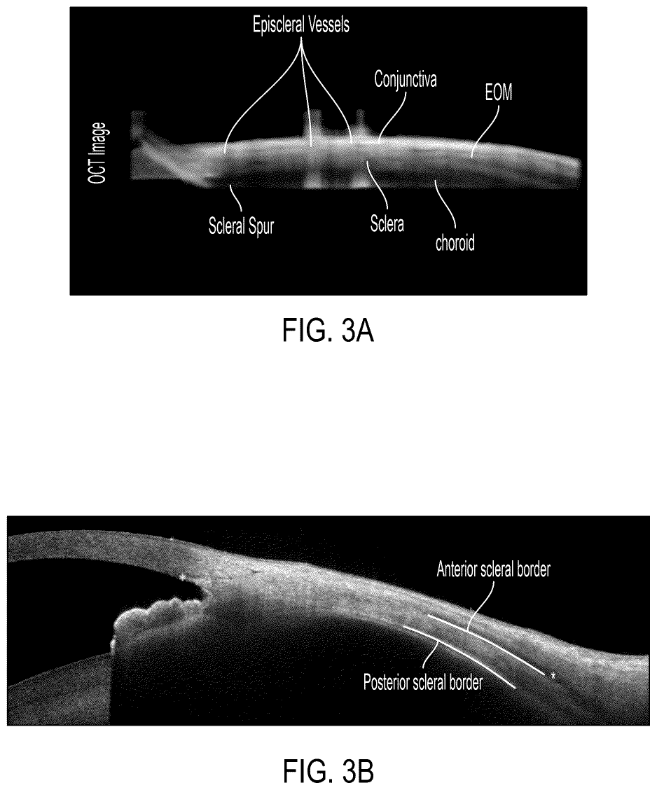

Instrument 100 may be used to administer a medicament to a target region of a subject's eye, such as the SCS. Prior to use, a thickness of a subject's sclera may be measured via, for example, anterior segment optical coherence tomography (OCT). Examples of OCT images are shown in FIGS. 3A and 3B. OCT images may be used, for example, to identify parts of the eye such as the episcleral vessels, conjunctiva, extraocular muscle (EOM), sclera, scleral spur, choroid, anterior scleral border, and posterior scleral border. OCT images may in turn be used to determine a thickness of the sclera. In some embodiments, a grayscale analysis of OCT imagery may be used to identify parts of the eye.

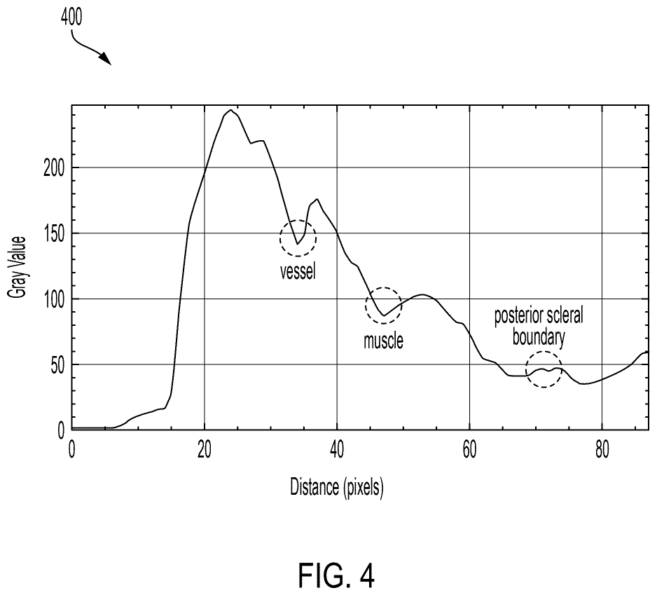

FIG. 4 illustrates a graphical representation 400 of such a grayscale analysis. In FIG. 4, the origin may represent an outer surface of the eye where the gray value is minimal, or 0. A local minimum in the gray value may exist at roughly 35 pixels below the surface, which may indicate an episcleral vessel. Another local minimum in the gray value may exist at approximately 47 pixels below the surface and may indicate EOM. A local maximum in the gray value may exist at approximately 70 pixels below the surface and may indicate a posterior scleral boundary. With a known resolution of the OCT imagery, the 70 pixels may be converted to a thickness measurement representing a thickness of the sclera.

In some embodiments, an instrument that is separate from instrument 100 may be used to obtain the thickness measurement. In some embodiments, the separate measuring instrument may be a specialized interferometry tool. In some embodiments, the separate measuring instrument may be a specialized ultrasound elastography tool. The measuring instrument may have a resolution of approximately 20 μm, 10 μm, or 5 μm. Such high resolution may allow for an accurate measurement of the scleral thickness.

Once the scleral thickness is known, instrument 100 may be adjusted such that needle 132 extends a corresponding length from eye shield 134. The length may be adjusted such that upon placement of eye shield 134 onto a subject's eye, needle 132 penetrates a sclera and enters an SCS, but does not penetrate a choroid or other layers of the eye. To adjust instrument 100, adjustment wheel 138 may be rotated relative to syringe body 110, eye shield 134, and/or needle collar 136. Rotation of adjustment wheel 138 may cause adjustment wheel 138 to translate axially relative to needle collar 136. Axial translation of adjustment wheel 138 may in turn cause eye shield 134 to translate axially relative to needle collar 136, due to contact between inner surfaces of chamber 144 and adjustment wheel 138. As a result, rotation of adjustment wheel 138 may alter a position of the distal tip of needle 132 relative to eye shield 134. Threaded portion 142 and the corresponding threads on the interior of adjustment wheel 138 may be fine pitch threads. Accordingly, fine adjustment of the position of the distal tip of needle 132 may be possible.

In use, with the distal tip of needle 132 set to a desired position, instrument 100 may be advanced toward a subject's eye such that needle 132 penetrates the sclera. A user may further advance instrument 100 until eye shield 134 comes in contact with the eye and flange 148 cups the surface of the eye. Eye shield 134 and flange 148 may inhibit further advancing of instrument 100 such that needle 132 is inhibited from advancing past an SCS into a choroid and/or other layers of the eye. In some embodiments, instrument 100 may be advanced such that an edge of flange 148 opposite cutout 150 first contacts the subject's eye. Instrument 100 may then be rotated forward until a front corner of flange 148 adjacent cutout 150 comes into contact with the subject's eye. While a distal tip of needle 132 is positioned within the SCS, medicament within barrel 112 may be delivered by depressing plunger 120. Instrument 100 may then be withdrawn from the eye.

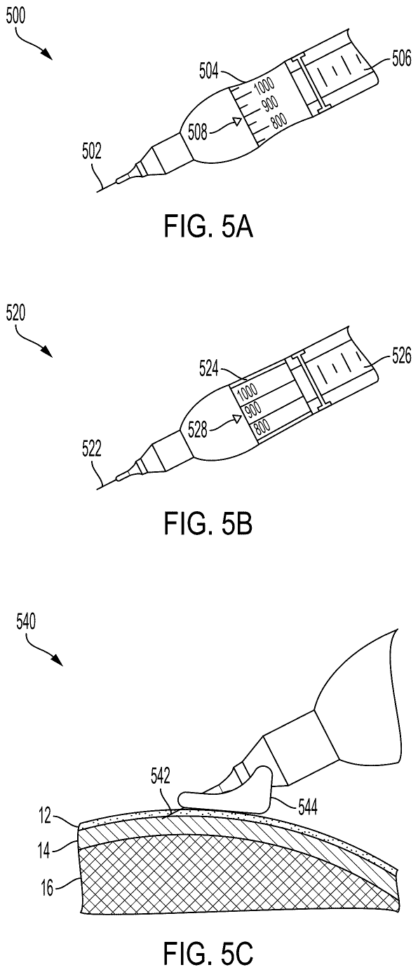

FIGS. 5A and 5B depict instruments similar to instrument 100. In FIG. 5A, an instrument 500 may include a needle 502, an adjustment wheel 504, a barrel 506, and an indicator marking 508. Adjustment wheel 504 may include numerical markings thereon indicating a position of the distal tip of needle 502 to a user. In some embodiments, linear markings similar to those found on a ruler or other markings may be included to indicate positions intermediate to numbered positions. In some embodiments, adjustment wheel 504 may be curved inwardly for ergonomic comfort.

Instrument 520, shown in FIG. 5B, may similarly include a needle 522, an adjustment wheel 524, a barrel 526, and an indicator marking 528. Adjustment wheel 524 may include numerical markings thereon that are separated extending axially along a length of adjustment wheel 524.

In some embodiments, adjustment wheels 138, 504, 524 may be continuously adjustable. In some embodiments, adjustment wheels 138, 504, 524 may be adjustable in discrete increments. In some embodiments, adjustment wheels 138, 504, 524 may lock to ensure that a desired position of the distal tip of the needle relative to the eye shield is maintained. In some embodiments, tactile feedback may be provided to the user to indicate locking. In some embodiments, disengagement of a locking mechanism may be required to adjust the position of the distal tip of the needle.

FIG. 5C illustrates an instrument 540, similar to any of instruments 100, 500, and/or 520, during delivery of medicament to an eye. Instrument 540 may include a needle 542 and an eye shield 544. Once a position of needle 542 is set relative to eye shield 544, as described previously, instrument 540 may be advanced toward a subject's eye such that needle 542 penetrates a sclera 12 of the eye. Instrument 540 may be further advanced until eye shield 544 comes into contact with sclera 12 and inhibits further movement of instrument 540. With eye shield 544 in contact with sclera 12, needle 542 may extend into SCS 14 without penetrating a choroid 16 or other layers of the eye. Instrument 540 may then be used to deliver medicament to an SCS 14.

Components of instruments 100, 500, 520, and/or 540 may be made of any suitable metal, polymer, and/or combination of metals and/or polymers. Exemplary metallic materials may include stainless steel, nitinol, titanium, and/or alloys of these metals. Exemplary polymeric materials may include polyetheretherketone (PEEK), polyimide, and polyethersulfone (PES). In some examples, components of instrument 100, 500, 520, and/or 540 may be made of a rigid material, semi-rigid material, or flexible material, wherein such material may be expandable and/or may allow for various configurations as discussed herein. Materials of instruments 100, 500, 520, and/or 540 may be any biocompatible material that may be sterilized. Unless otherwise mentioned, similar materials may be incorporated in embodiments described hereinafter.

Instruments 500, 520, and 540 are shown partially in FIGS. 5A-5C and it should be understood that instruments 500, 520, and 540 may include other components similar to those of instrument 100, such as syringe bodies and a plungers for expelling medicament through the respective needles. Additionally, in some embodiments, instruments 100, 500, 520, and/or 540 may be single-use disposable devices. In some embodiments, instruments 100, 500, 520, and/or 540 may be reusable after re-filling and/or re-sterilization.

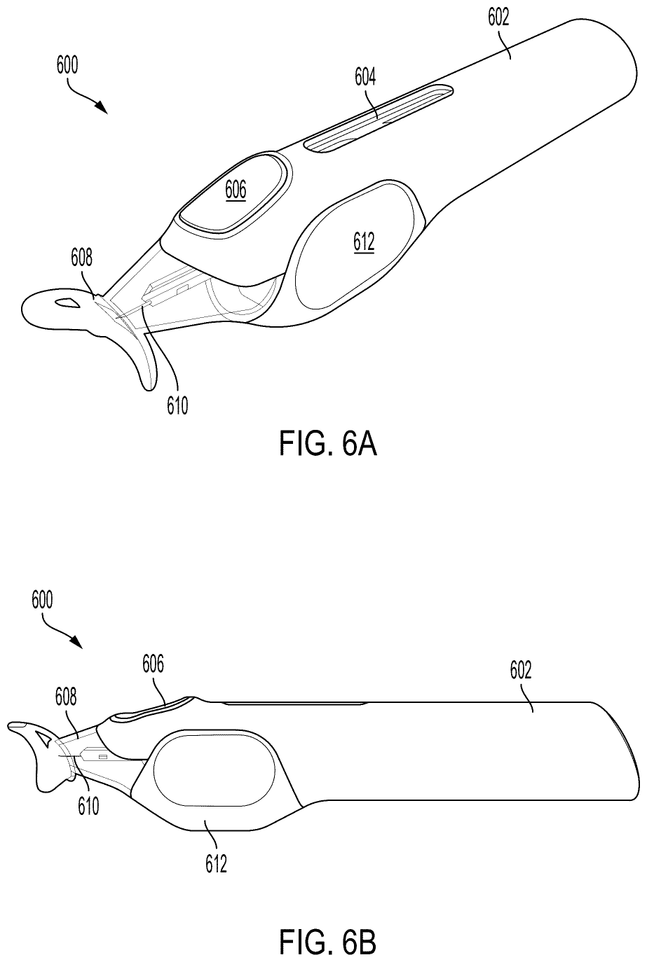

FIGS. 6A and 6B depict another exemplary instrument 600 in accordance with the present disclosure. Instrument 600 may include a housing 602, a window 604, an activation button 606, an eye shield 608, and a needle 610. In some embodiments, instrument 600 may further include a grip portion 612 of housing 602. Grip portion 612 may be dimensioned to enhance a user's comfort and/or control of instrument 600. During use, eye shield 608 may be configured to be placed against a sclera of a subject's eye. Eye shield 608 may be configured such that placement of eye shield 608 against a subject's eye orients and maintains needle 610 at an acute angle relative to a tangent of an eye surface.

Prior to use, distal tip of needle 610 may protrude a predetermined length from eye shield 608. Needle 610 may, for example, protrude between 100 μm and 1 mm, between 300 μm and 800 μm, or between 400 μm and 700 μm beyond eye shield 608 prior to use. In some embodiments, needle 610 may protrude about 400 μm, about 500 μm, or about 600 μm beyond eye shield 608 prior to use. In embodiments in which needle 610 protrudes beyond eye shield 608 prior to use, eye shield 608 may be placed against a subject's eye when performing an injection, causing needle 610 to penetrate an outer surface of the eye slightly. In some embodiments, needle 610 may not protrude beyond eye shield 608 prior to use.

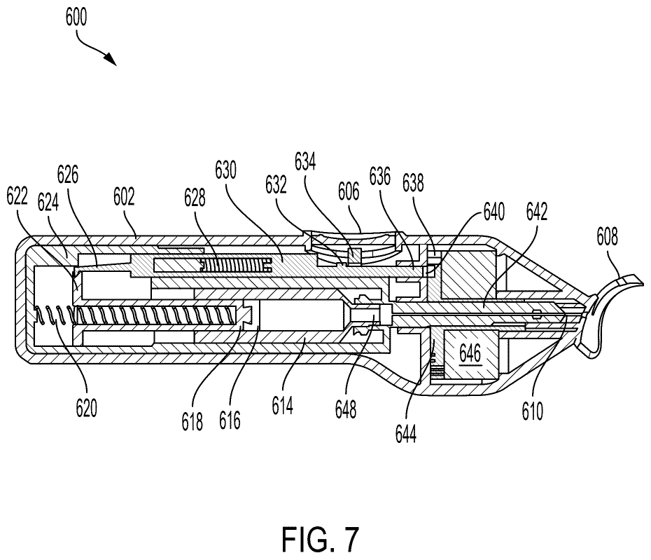

FIG. 7 illustrates a cross-sectional view of instrument 600. Within housing 602, instrument 600 may include a cartridge 614, a stopper 616, a plunger 618, a biasing mechanism 620, and a carriage 624. Instrument 600 may further include a spring rod 630, a rotary driver 638, a leadscrew nut 644, and a damper 646. Cartridge 614 may be inserted within and coupled to carriage 624. Cartridge 614 may be a standard luer-type vial, for example, and may include a neck 648, which may be coupled to a needle overmold 642 of carriage 624. Needle overmold 642 may retain needle 610 and may form a fluid conduit between cartridge 614 and needle 610.

Instrument 600 may configured such that needle 610 may advance into a subject's SCS and medicament from cartridge 614 may be delivered via needle 610 upon pressing of activation button 606 by a user. A needle drive mechanism, for example, may cause needle 610 to advance into a subject's SCS. Leadscrew nut 644, rotary driver 638, needle overmold 642, and/or damper 646 may collectively form the needle drive mechanism. During operation, rotary driver 638 may apply a torque to leadscrew nut 644, causing leadscrew nut 644 to rotate about needle overmold 642. In some embodiments, rotary driver 638 may be a torsion spring, a clock spring, or any other known device configured to apply a torque to leadscrew nut 644. In some embodiments, rotary driver 638 may be configured to apply a constant torque to leadscrew nut 644.

Leadscrew nut 644 may engage with needle overmold 642 via threading such that needle overmold 642 acts as a leadscrew and advances distally as leadscrew nut 644 rotates, thereby causing needle 610 to advance distally. In particular, needle overmold 642 may include threading on an outer surface thereof while leadscrew nut 644 may include threading on an inner surface thereof. Damper 646 may dampen rotation of rotary driver 638 and/or leadscrew nut 644 such that needle overmold 642 advances smoothly and/or at a substantially constant rate. In some embodiments, damper 646 may be a rotary damper, a gear damper, a barrel damper, a disc damper, a continuous angle damper, a limited angle damper, or any other suitable form of damper. In some embodiments, needle overmold 642 may be coupled to or integrally formed with carriage 624 such that advancement of needle overmold 642 results in advancement of carriage 624 and components coupled thereto.

Prior to use, rotary driver 638 may be maintained in an energized or compressed configuration by stop 636. Stop 636 may initially extend through opening 640 into a rotational path of leadscrew nut 644 and/or rotary driver 638 to impede rotation of leadscrew nut 644 and/or rotary driver 638. When activation button 606 is depressed by a user to activate instrument 600, protrusion 634 may cause lug 632 to move in a proximal direction. Movement of lug 632 may in turn cause spring rod 630 to move proximally against a force applied by biasing mechanism 628, thereby withdrawing stop 636 from the rotational path of leadscrew nut 644 and/or rotary driver 638. In some embodiments, biasing mechanism 628 may be an axial spring that is compressed upon depression of activation button 606. Biasing mechanism 628 may be coupled at one end to carriage 624 and coupled at an opposing end to spring rod 630. Withdrawal of stop 636 may release leadscrew nut 644 and/or rotary driver 638 and allow rotary driver 638 to transition to an expanded configuration.

Before needle 610 advances to a subject's SCS, fluid pressure exerted by scleral tissue may inhibit stopper 616 from advancing. However, when needle 610 advances to a subject's SCS, the fluid pressure may decrease and biasing mechanism 620 may advance plunger 618 and stopper 616 within cartridge 614. As a result, medicament may be discharged from cartridge 614 to the SCS via needle 610. In some embodiments, biasing mechanism 620 may be an axial spring initially in compression. An expanded length of the axial spring may be selected such that force applied to plunger 618 by the spring may be substantially constant over a travel distance of stopper 616. Biasing mechanism 620 may be coupled at one end to carriage 624 and coupled at an opposing end to plunger 618. The reduction in fluid pressure on contents of cartridge 614 may allow biasing mechanism 620 to expand, thereby forcing the contents of cartridge 614 through needle 610 and into the SCS. In some embodiments, biasing mechanism 620 may be activated by pressing activation button 606 and may be inhibited from advancing until activation button 606 is pressed.

Plunger 618 may include a flange 622 that extends perpendicularly to an axis of travel of plunger 618. Prior to movement of plunger 618, flange 622 may interfere with a finger 626 of spring rod 630 such that flange 622 and spring rod 630 collectively resist a force of biasing mechanism 628 applied to spring rod 630 toward opening 640. When plunger 618 advances within cartridge 614 a predetermined distance, finger 626 may release from flange 622, allowing biasing mechanism 628 to advance spring rod 630 toward opening 640 such that stop 636 impedes further rotation of leadscrew nut 644 and/or rotary driver 638. Further advancing of needle 610 into the subject's eye may effectively cease in response to advancing of plunger 618 and stopper 616 through cartridge 614. In some embodiments, plunger 618 and stopper 616 may be configured to continue advancing and discharging medicament from cartridge 614.

Progress of an injection may be visible to a user through window 604. For example, a user may be able to view movements of plunger 618 and/or stopper 616 through window 604. A user may also be able to view an amount of medicament remaining in cartridge 614 through window 604. A speed of movement of plunger 618 and/or stopper 616 through cartridge 614 may depend on several variable factors, include a viscosity of the medicament, a length and diameter of needle 610, a gauge of biasing mechanism 620, or other factors. The speed of movement of plunger 618 and/or stopper 616 through cartridge 614 may also depend on an IOP of a subject's eye.

To perform an injection and administer a medicament using instrument 600, a user may first insert cartridge 614 into housing 602. Instrument 600 may be configured such that closing housing 602 after inserting cartridge 614 primes a fluid pathway extending from cartridge 614 through needle 610. The user may then place eye shield 608 against an outer surface of a subject's eye. In embodiments in which needle 610 extends beyond eye shield 608 prior to use, placement of 608 against the outer surface of the subject's eye may cause needle 610 to penetrate the eye slightly, positioning the distal tip of needle 610 within a sclera of the eye. Additionally, placement of eye shield 608 against the outer surface of the eye such that a curvature of eye shield 608 conforms to the eye may orient needle 610 at a 45° angle relative to a tangent of the outer surface.

If the user and/or subject are satisfied with the positioning of instrument 600 relative to the subject's eye, the user may depress activation button 606. As a result, stop 636 may withdraw from impeding rotation of rotary driver 638 and/or leadscrew nut 644. In some embodiments, depression of activation button 606 may activate rotary driver 638 without causing stop 636 to withdraw from rotary driver 638. Rotary driver 638 may then cause leadscrew nut 644 to rotate about needle overmold 642. Due to the threading between leadscrew nut 644 and needle overmold 642, such rotation may cause needle overmold 642 and needle 610 to advance distally relative to instrument 600 and further into the subject's eye. While the distal tip of needle 610 is within the sclera of the subject's eye, fluid pressure acting on the contents of cartridge 614 may be sufficiently high to resist an axial force of biasing mechanism 620 and prevent stopper 616 from advancing forward.

When the distal tip of needle 610 passes through the sclera and enters the SCS, fluid pressure acting on the contents of cartridge 614 may decrease sufficiently to allow biasing mechanism 620 to overcome a force exerted against stopper 616 by the contents of cartridge 614 in a proximal direction and advance plunger 618 and stopper 616 distally within cartridge 614. As stopper 616 advances distally, medicament within cartridge 614 may be discharged via needle 610 into the SCS.

When plunger 618 has advanced a predetermined distance within cartridge 614, finger 626 may become decoupled from flange 622 of plunger 618. Without a force exerted by flange 622 on finger 626 to counteract a force exerted by biasing mechanism 628 on spring rod 630, biasing mechanism 628 may then urge spring rod 630 distally, such that stop 636 advances into the rotational path of rotary driver 638 and/or leadscrew nut 644. Stop 636 may then impede rotation of rotary driver 638 and/or leadscrew nut 644 and needle 610 may cease advancing into the subject's eye.

Stopper 616 may continue to advance through cartridge 614 to discharge medicament via needle 610. The user may observe the position of stopper 616 and/or an amount of medicament remaining within cartridge 614 through window 604. When stopper 616 reaches an end of cartridge 614 or a desired amount of medicament has otherwise been discharged, the user may remove instrument 600 from the subject's eye, thereby withdrawing needle 610 and terminating the injection procedure.

Instrument 600 may therefore provide an easily operable device for administering medicament to a subject's eye. A user may successfully administer medicament simply by placing instrument 600 in a desired position relative to a subject's eye and pressing a button. The simplicity of operation may promote repeatability of safe injections and comfort for subjects. Additionally, in some embodiments, instrument 600 may be a single-use disposable device. In some embodiments, instrument 600 may be reusable after re-filling, replacing the cartridge, and/or re-sterilization.

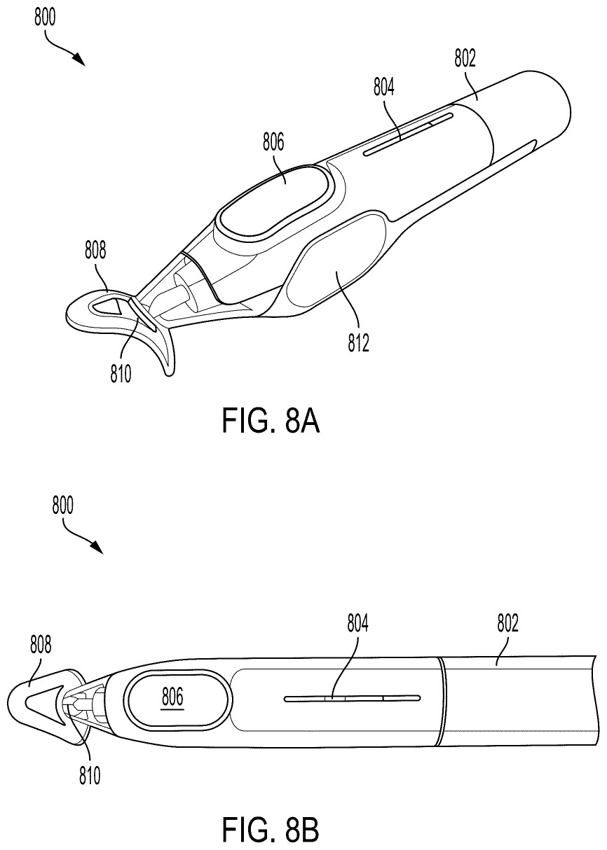

FIGS. 8A and 8B depict another exemplary instrument 800 in accordance with the present disclosure. Instrument 800 may be an electronic drug delivery device and may include a housing 802, a window 804, an activation button 806, an eye shield 808, and a needle 810. In some embodiments, window 804 may allow a user to see internal components of instrument 800 to monitor progress of an injection. Additionally or alternatively, in some embodiments, window 804 may facilitate a different visual indication of injection progress, such as illumination of LED lights. In some embodiments, instrument 800 may further include a grip portion 812 of housing 802. Grip portion 812 may be dimensioned to enhance a user's comfort and/or control of instrument 800. During use, eye shield 808 may be configured to be placed against a sclera of a subject's eye. Eye shield 808 may be configured such that placement of eye shield 808 against a subject's eye orients and maintains needle 810 at 45° relative to a tangent of an eye surface.

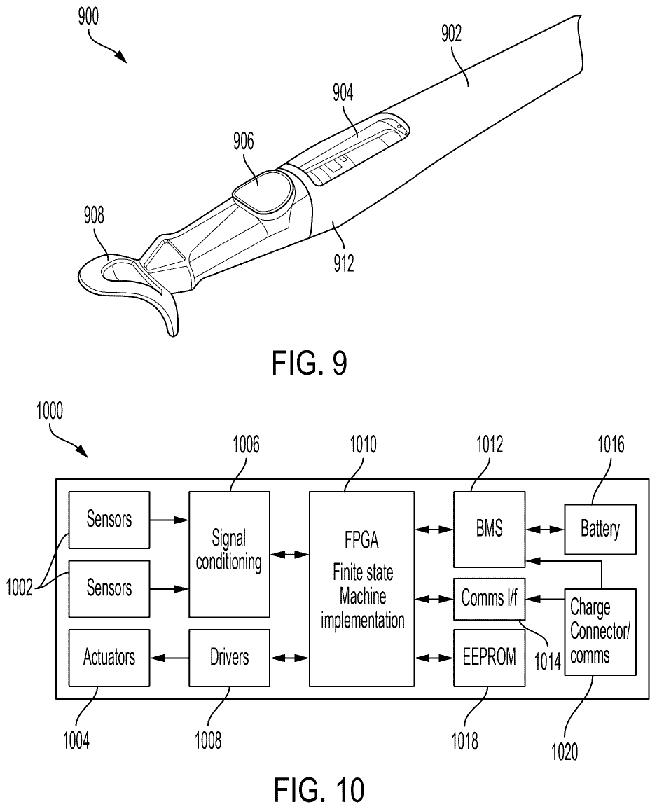

FIG. 9 depicts an instrument 900 similar to instrument 800 having a slightly different form factor. Instrument 900 may include a housing 902, a window 904, an activation button 906, an eye shield 908, and a needle (not shown). In some embodiments, window 904 may allow a user to see internal components of instrument 900 to monitor progress of an injection. Additionally or alternatively, in some embodiments, window 904 may facilitate a different visual indication of injection progress, such as illumination of LED lights. Instrument 900 may further include a narrowed grip portion 912 of housing 902. Grip portion 912 may be dimensioned to reduce an overall profile of instrument 900 while maintaining ergonomic benefits. Eye shield 908 may similarly be configured such that placement of eye shield 908 against a subject's eye orients and maintains the needle at 45° relative to a tangent of an eye surface.

FIG. 10 illustrates a block diagram 1000 representing a schematic of electronic components of instrument 800 and/or instrument 900. Instrument 800 and/or instrument 900 may include a plurality of sensors 1002. Sensors 1002 may include sensors for detecting contact between eye shield 808 or eye shield 908 and a subject's eye. Sensors 1002 may further include force sensors, pressure sensors, temperature sensors, or other suitable sensors. Signals generated by sensors 1002 may be transmitted to a signal conditioning block 1006, in which the signals may be filtered, amplified, mixed, multiplexed, or otherwise conditioned for processing by a logic element. The conditioned signals may be transmitted to field programmable gate array (FPGA) 1010. FPGA 1010 may be implemented as a finite state machine such that instrument 800 and/or instrument 900 may have a limited number of states and may be in only one state at a time. Instrument 800 and/or instrument 900 may, for example, have an state in which the instrument is actively performing an injection and a deactivated state in which the instrument is not performing an injection.

FPGA 1010 may be programmed to transmit signals to, and receive signals from, drivers 1008, which in turn control actuators 1004. Actuators 1004 may include electric motors, pumps, solenoids, or other types of actuators. FPGA 1010 may cause actuators 1004, via drivers 1008, to perform a corresponding function in response to signals received from sensors 1002, for example.

Instrument 800 and/or instrument 900 may further include a battery management system (BMS) 1012, a communications interface 1014, an electrically erasable programmable read-only memory (EEPROM) 1018, a battery 1016, and a charger/communications port 1020. In some embodiments, battery 1016 may be 3V lithium ½ AA battery. BMS 1012 may communicate with FPGA 1010 and battery 1016 and may include logic for extending a life of battery 1016 and/or preserving a charge of battery 1016. BMS 1012 may further receive power from charge/communications port 1020, which may be supplied by an external power supply. BMS 1012 may regulate power supplied to battery 1016 for charging. Charge/communications port 1020 may also allow for connection of external devices to instrument 800 and/or instrument 900. Such external devices may transmit data to, or receive data from, FPGA 1010 via communications interface 1014. EEPROM 1018 may be used to store instructions, routines, and/or data values accessed by FPGA 1010.

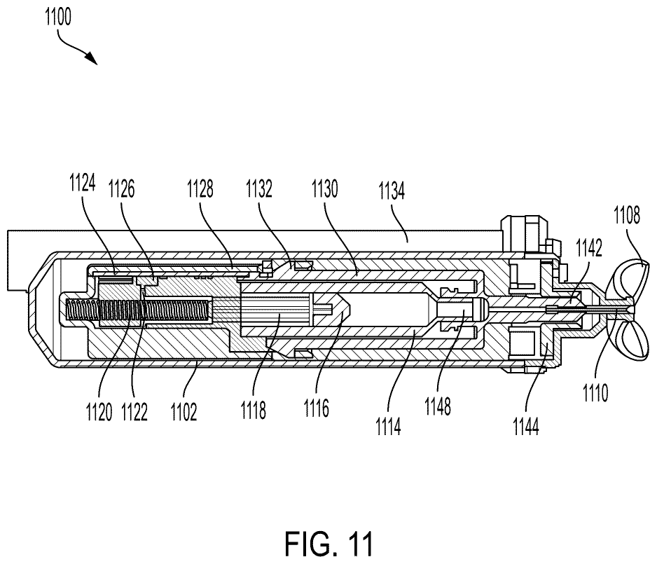

FIG. 11 illustrates a cross-sectional view of an instrument 1100 that may include substantially similar components as instrument 800 and/or instrument 900. Instrument 1100 may include a housing 1102 containing a cartridge 1114, an eye shield 1108, a stopper 1116, a plunger 1118, a biasing mechanism 1120, and a proximal carriage 1128 and a distal carriage 1130. Distal carriage 1130 may include one or more protrusions 1132 configured to extend into corresponding openings of proximal carriage 1128, thereby coupling distal carriage 1130 with proximal carriage 1128. In some embodiments, eye shield 1108 may include one or more contact sensors for detecting contact with a subject's eye. Instrument 1100 may further include a printed circuit board (PCB) 1124, a microswitch 1126, a battery compartment 1134, and a leadscrew nut 1144. PCB 1124 may include and/or function as a controller for instrument 1100. In some embodiments, as described previously with reference to FIG. 10, the controller may be a FPGA like FPGA 1010. Cartridge 1114 may be inserted within and coupled to distal carriage 1130. Cartridge 1114 may include a neck 1148 which is coupled to a needle overmold 1142 of distal carriage 1130. Needle overmold 1142 may retain needle 1110 and may form a fluid conduit between cartridge 1114 and needle 1110. Battery compartment 1134 may be configured to retain one or more batteries for supplying power to instrument 1100.

Instrument 1100 may be configured such that needle 1110 may advance into a subject's SCS and medicament from cartridge 1114 may be delivered via needle 1110 upon placement of eye shield 1108 on a subject's eye and pressing of an activation button, such as activation button 806 or activation button 906, by a user. A needle drive mechanism may cause needle 1110 to advance into a subject's SCS. Leadscrew nut 1144, a motor (not shown) to drive leadscrew nut 1144, and/or needle overmold 1142 may collectively form the needle drive mechanism. During operation, the motor may apply a torque to leadscrew nut 1144, causing leadscrew nut 1144 to rotate about needle overmold 1142. In some embodiments, the motor may be an electric motor, a geared motor, or any other suitable motor configured to selectively apply a torque to leadscrew nut 1144. In some embodiments, the motor may be configured to apply a constant torque to leadscrew nut 1144. Leadscrew nut 1144 may engage with needle overmold 1142 via threading such that needle overmold 1142 acts as a leadscrew and advances distally as leadscrew nut 1144 rotates, thereby causing needle 1110 to advance distally. In particular, needle overmold 1142 may include threading on an outer surface thereof while leadscrew nut 1144 may include threading on an inner surface thereof.

When eye shield 1108 is placed against a subject's eye and the activation button is depressed by a user, PCB 1124 may receive one or more signals indicative of the placement of eye shield 1108 and depression of the activation button. In response to the one or more signals, PCB 1124 may cause the motor to apply a torque to leadscrew nut 1144. Needle 1110 may then advance distally into the subject's eye. In some embodiments, PCB 1124 may be configured to cause an actuator (not shown) to release biasing mechanism 1120 when needle 1110 has advanced a predetermined distance. In some embodiments, the predetermined distance may be between 100 μm and 1 mm, between 300 μm and 800 μm, between 400 μm and 700 μm beyond eye shield 608 prior to use. In some embodiments, the predetermined distance may be about 400 μm, about 500 μm, or about 600 μm.

After biasing mechanism 1120 is released and before needle 1110 advances to a subject's SCS, fluid pressure exerted by scleral tissue may inhibit stopper 1116 from advancing. However, when needle 1110 advances to a subject's SCS, the fluid pressure may decrease and biasing mechanism 1120 may advance plunger 1118 and stopper 1116 within cartridge 1114. As a result, medicament may be discharged from cartridge 1114 to the SCS via needle 1110. In some embodiments, biasing mechanism 1120 may be an axial spring initially in compression. An expanded length of the axial spring may be selected such that force applied to plunger 1118 by the spring may be substantially constant over a travel distance of stopper 1116. Biasing mechanism 1120 may be coupled at one end to proximal carriage 1128 and coupled at an opposing end to plunger 1118 The reduction in fluid pressure on contents of cartridge 1114 may allow biasing mechanism 1120 to expand, thereby forcing the contents of cartridge 1114 through needle 1110 and into the SCS.

Plunger 1118 may include a flange 1122 that extends perpendicularly to an axis of travel of plunger 1118. Prior to use, flange 1122 may be proximally relative to microswitch 1126. When plunger 1118 advances within cartridge 1114 a predetermined distance, flange 1122 may toggle microswitch 1126. PCB 1124 may receive a signal indicating that microswitch 1126 has been toggled. In response, PCB 1124 may cause the motor to cease applying torque to leadscrew nut 1144. Further advancing of needle 1110 into the subject's eye may effectively cease in response to advancing of plunger 1118 and stopper 1116 through cartridge 1114. As a result, advancing of needle 1110 may be limited to reaching the SCS, but not penetrating deeper layers of the eye. In some embodiments, plunger 1118 and stopper 1116 may be configured to continue advancing and discharging medicament from cartridge 1114.

Though not shown in FIG. 11, a second microswitch may be positioned distally relative to microswitch 1126. The second microswitch may be configured to detect movement of flange 1122 to a position corresponding to complete translation of stopper 1116 to a distal end of cartridge 1114. PCB 1124 may receive a signal indicating that the second microswitch has been toggled when flange 1122 reaches the position. In response, PCB 1124 may cause the motor to apply a torque to leadscrew nut 1144 in an opposite direction, thereby withdrawing needle 1110 from the subject's eye.

Progress of an injection may be visible to a user through window, such as window 804 and/or window 904 described previously. For example, a user may be able to view movements of plunger 1118 and/or stopper 1116 through the window. A user may also be able to view an amount of medicament remaining in cartridge 1114 through the window. A speed of movement of plunger 1118 and/or stopper 1116 through cartridge 1114 may depend on several variable factors, including a viscosity of the medicament, a length and diameter of needle 1110, a gauge of biasing mechanism 1120, or other factors. The speed of movement of plunger 1118 and/or stopper 1116 through cartridge 1114 may also depend on an IOP of a subject's eye.

Additionally, or alternatively, a visual proxy for injection progress may be visible through the window. For example, one or more light emitting diodes may be controlled by PCB 1124 to illuminate at predetermined stages of an injection.

To perform an injection and administer a medicament using instrument 1100, a user may first insert cartridge 1114 into housing 1102. Instrument 1100 may be configured such that closing housing 1102 after inserting cartridge 1114 primes a fluid pathway extending from cartridge 1114 through needle 1110. The user may then place eye shield 1108 against an outer surface of a subject's eye. Placement of eye shield 1108 against the outer surface of the eye such that a curvature of eye shield 1108 conforms to the eye may orient needle 1110 at a 45° angle relative to a tangent of the outer surface.

If the instrument 1100 is positioned properly relative to the subject's eye, contact sensors within eye shield 1108 may transmit corresponding signals to PCB 1124. Subsequent depression of the activation button, in combination with signals from the contact sensors, may trigger PCB 1124 to drive the motor. The motor may then cause leadscrew nut 1144 to rotate about needle overmold 1142. Due to the threading between leadscrew nut 1144 and needle overmold 1142, such rotation may cause needle overmold 1142 and needle 1110 to advance distally relative to housing 1102 such that needle 1110 penetrates into the subject's eye. After needle 1110 advances a predetermined distance, PCB 1124 may cause an actuator to release biasing mechanism 1120. While the distal tip of needle 1110 is within the sclera of the subject's eye, fluid pressure acting on the contents of cartridge 1114 may be sufficiently high to resist an axial force of biasing mechanism 1120 and prevent stopper 1116 from advancing distally.

When the distal tip of needle 1110 passes through the sclera and enters the SCS, fluid pressure acting on the contents of cartridge 1114 may decrease sufficiently to allow biasing mechanism 1120 to overcome a force exerted against stopper 1116 by the contents of cartridge 1114 in a proximal direction and advance plunger 1118 and stopper 1116 distally within cartridge 1114. As stopper 1116 advances distally, medicament within cartridge 1114 may be discharged via needle 1110 into the SCS.

When plunger 1118 has advanced a predetermined distance within cartridge 1114, microswitch 1126 may be toggled by flange 1122. In response, PCB 1124 may cause the motor to cease applying torque to leadscrew nut 1144. Needle 1110 may cease advancing into the subject's eye.

Stopper 1116 may continue to advance through cartridge 1114 to discharge medicament via needle 1110 as biasing mechanism 1120 continues to expand. The user may observe the position of stopper 1116 and/or an amount of medicament remaining within cartridge 1114 through the window. Additionally, or alternatively, the user may view LED indications of progress through the window. When stopper 1116 reaches an end of cartridge 1114 or a desired amount of medicament has otherwise been discharged, the second microswitch may be toggled. In response, PCB 1124 may cause the motor to drive leadscrew nut 1144 in reverse, withdrawing needle 1110 from the eye. The user may then remove instrument 1100 from the subject's eye, thereby terminating the injection procedure.

Instruments 800, 900, and/or 1100 may therefore provide an easily operable device for administering medicament to a subject's eye. A user may successfully administer medicament simply by placing instruments 800, 900, and/or 1100 in a desired position relative to a subject's eye and pressing a button. The simplicity of operation may promote repeatability of safe injections and comfort for subjects. Additionally, in some embodiments, instruments 800, 900, and/or 1100 may be single-use disposable devices. In some embodiments, instruments 800, 900, and/or 1100 may be reusable after re-filling, replacing the cartridge, and/or re-sterilization.

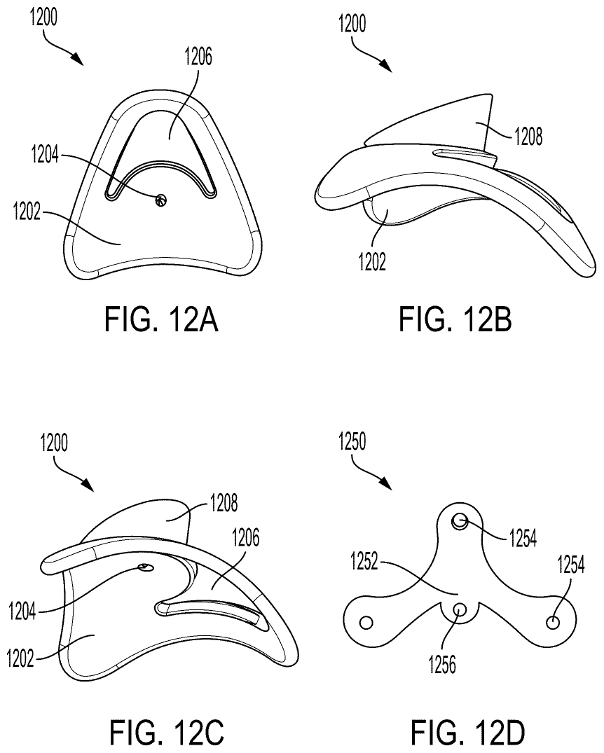

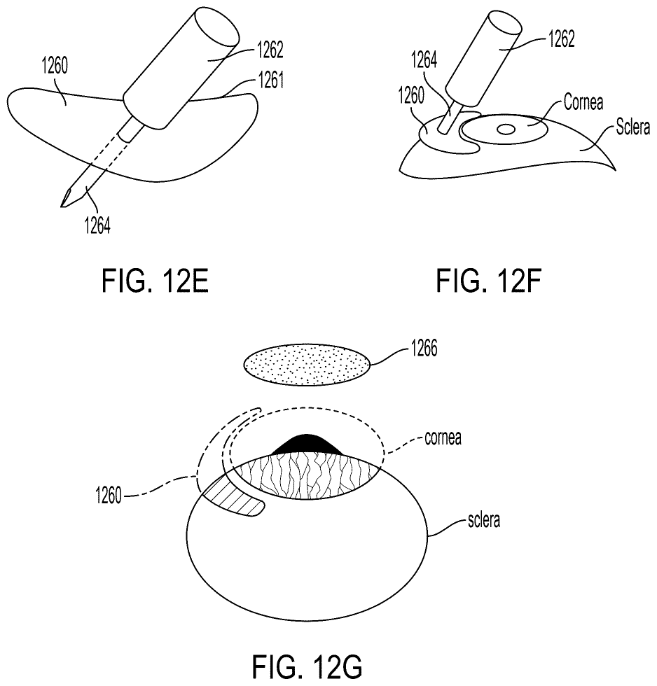

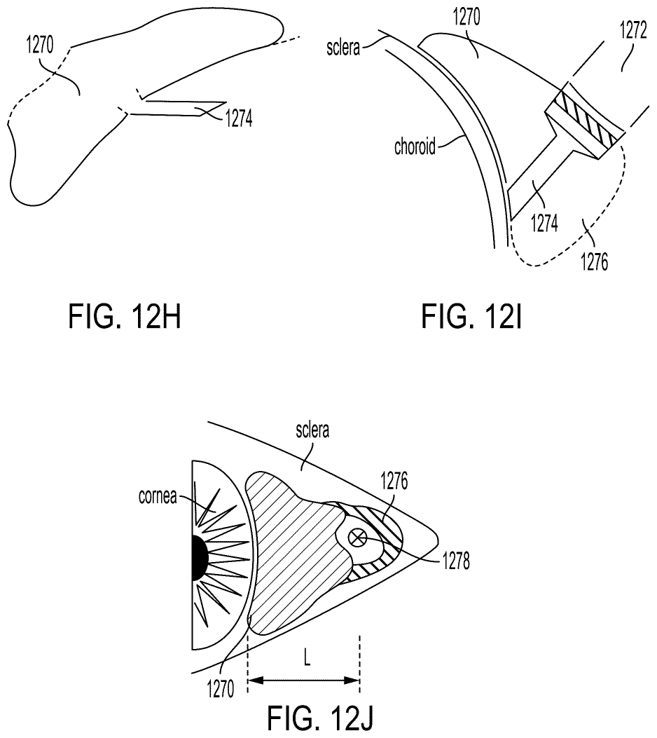

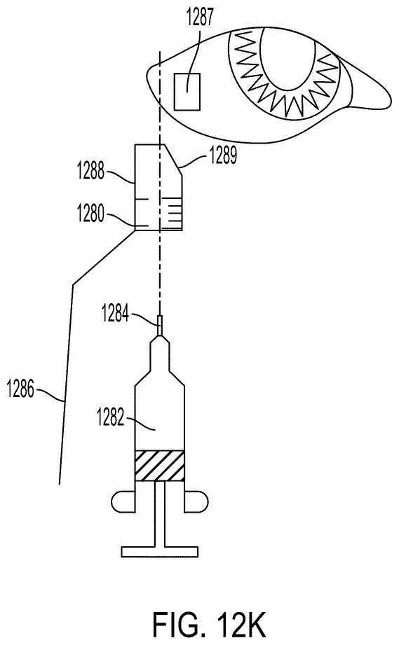

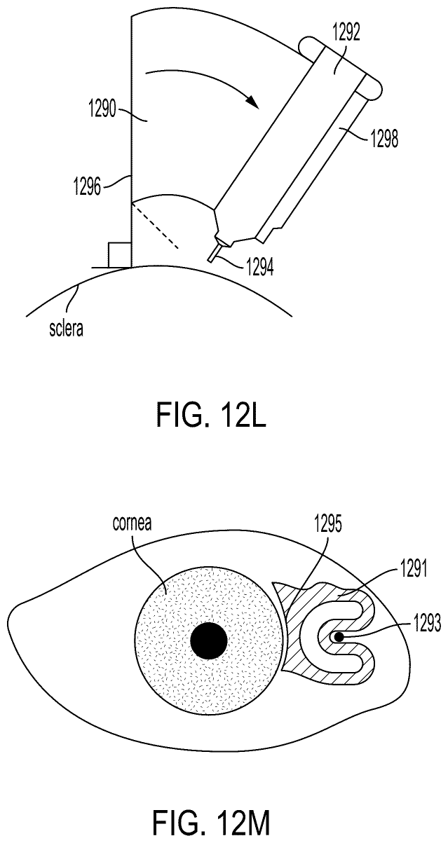

FIGS. 12A-12D illustrate an eye shield and contact sensors that may be used with embodiments of the disclosure and/or substituted for the eye shields previously described. An eye shield 1200 may include a contoured surface 1202 configured to mate with an outermost surface of a subject's eye. Eye shield 1200 may further include a through-hole 1204 and a cutout 1206. Through-hole 1204 may be configured to allow a needle to pass through eye shield 1200. Through-hole 1204 may also be located to ensure that eye shield 1200 is positioned appropriately with respect to the needle. Cutout 1206 may allow eye shield 1200 to flex when positioned against a subject's eye to promote comfort. Eye shield 1200 may further include a collar 1208. Collar 1208 may be configured to couple to a distal end of a housing of an instrument.

In some embodiments, a sensor array 1250 may be included with or coupled to eye shield 1200. Sensor array 1250 may be positioned on contoured surface 1202, for example, and may include a body 1252, a plurality of sensors 1254, and a through-hole 1256. Through-hole 1256 may be aligned with through-hole 1204 to allow a needle to pass through both through-hole 1256 and through-hole 1204. The alignment may also ensure that sensors 1254 are positioned appropriately relative to the needle.

Sensors 1254 may be contact sensors configured to detect contact with an outer surface of a subject's eye. In some embodiments, sensors 1254 may be conductivity sensors, for example. In some embodiments, sensor array 1250 may further include one or more force sensors configured to detect a force applied to sensor array 1250 and/or eye shield 1200. In some embodiments, sensors 1254 may include capacitance sensors, moisture sensors, temperature sensors, or combinations thereof.

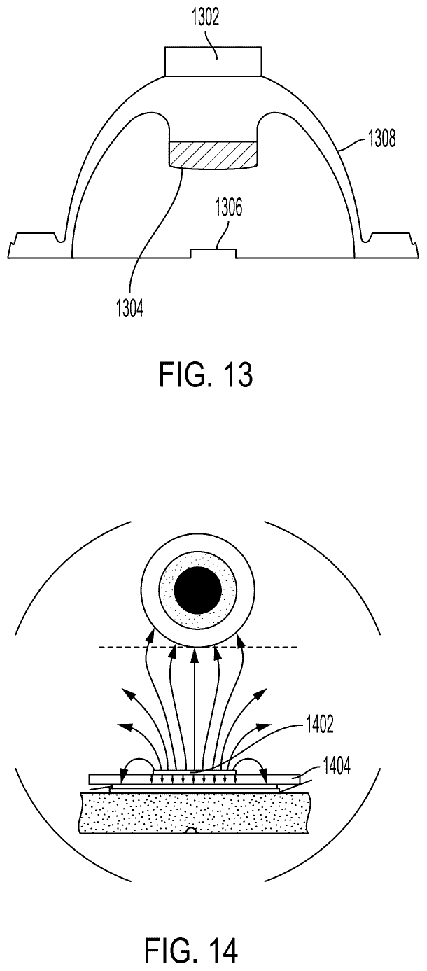

In some embodiments, eye shield 1200 may additionally or alternatively incorporate still other types of sensors. In some embodiments, eye shield 1200 may include ultrasonic sensors and/or optical sensors for detecting proximity to a sclera of a subject's eye. In some embodiments, eye shield 1200 may include a silicone dome switch, as shown in FIG. 13. The dome switch may include a compression surface 1302, a first contact 1304, a second contact 1306, and a deformable wall 1308. The dome switch may be configured such that first contact 1304 comes into contact with second contact 1306 when a desired force is applied to compression surface 1302 by a sclera. In some embodiments, eye shield 1200 may include a capacitive sensor 1402, as shown in FIG. 14. Capacitive sensor 1402 may be implemented directly on a dedicated PCB 1404 positioned on eye shield 1200, or may otherwise be electrically coupled to a remote PCB, like PCB 1124.

Eye shield 1200 and/or sensor array 1250, in combination with an instrument such as instrument 800, instrument 900, and/or instrument 1100, may assist a user with achieving correct alignment of the instrument with a subject's eye. Eye shield 1200 and/or sensor array 1250 may further assist a user with distributing force over a subject's eye, reducing a likelihood of pinching or excessive scleral compression. Eye shield 1200 and/or sensor array 1250 may further assist with maintaining a correct angle of a needle relative to a subject's eye. In some embodiments, eye shield 1200 may include a visible reference marking thereon. The reference marking may be positioned to align with an anatomical feature during placement of eye shield 1200. For example, reference marking may be configured to align with a cornea, a corner of an eyelid, a pupil, or any other suitable anatomical feature. Alignment of the reference marking may indicate to the user that eye shield 1200 is appropriately positioned.

Eye shields having other geometries and configurations may be used with the instruments of this disclosure. FIGS. 12E-12M illustrate further examples of such eye shields. FIG. 12E, for example, shows an eye shield 1260 that may be generally crescent-shaped. Eye shield 1260 may be configured to couple to a distal end of an instrument 1262 having a needle 1264. Eye shield 1260 may include a curved edge 1261 configured to conform to an outer portion of a cornea.

FIG. 12F illustrates an example orientation of eye shield 1260 and instrument 1262 relative to an eye. Eye shield 1260 may be positioned adjacent to an outer portion of a cornea. In use, eye shield 1260 may be placed on a sclera such that the curvature of the eye shield 1260 aligns with the outer portion of the cornea. In this position, a portion of instrument 1262 may be oriented above the cornea. Instrument 1262 may further intersect eye shield 1260 such that needle 1264 is positioned for nearly tangential penetration of the sclera. The cornea may thus guide positioning of eye shield 1260 and needle 1264 for insertion to a desired position and at a desired angle relative to the surface of the sclera. Additionally, the crescent shape of eye shield 1260 may minimize a surface area of the sclera contacted by eye shield 1260, thereby reducing patient discomfort.

In some embodiments, as shown in FIG. 12G, eye shield 1260 may be used together with a visual aid 1266. A patient may be instructed to focus his or her gaze on visual aid 1266 to maintain a consistent orientation of the cornea. By stabilizing the cornea, eye shield 1260 may also be stabilized to promote precise needle insertion.

FIGS. 12H-12I illustrate another variation of an eye shield. As shown in FIG. 12H, eye shield 1270 may be configured for use with a needle 1274. As shown in FIG. 12I, eye shield 1270 may be configured to couple to a distal end of an instrument 1272 incorporating needle 1274. Eye shield 1270 may include a curved distal surface configured to conform to a sclera of an eye. Eye shield 1270 may further be configured to direct needle 1274 at an angle relative to an orthogonal of the scleral surface. In some embodiments, the angle may be selected such that needle 1274 is positioned for nearly tangential penetration of the sclera.

In some embodiments, eye shield 1270 may be positioned to a side of needle 1274 and may be configured to partially surround needle 1274 about its axis such that needle 1274 is at least partially exposed about its axis. In some embodiments, eye shield 1270 may include a shield portion 1276 configured to surround needle 1274 about its axis.

FIG. 12J illustrates a footprint of eye shield 1270 on an eye. Similar to eye shield 1260, eye shield 1270 may include a surface configured to conform to a curvature of an outer portion of a cornea. The cornea-adjacent surface may be a distance L from an injection site 1278 to which eye shield 1270 directs needle 1274. As seen in FIG. 12J, eye shield 1270 may guide positioning of needle 1274 laterally of the cornea, using the cornea for alignment.

FIG. 12K illustrates another variation of an eye shield. As shown, eye shield 1280 may include a receiver portion 1288 and a handle portion 1286. Receiver portion 1288 may be configured to receive and/or hold a distal portion of an instrument 1282. Receiver portion may include a contact surface 1289 configured to be placed against a sclera of an eye. In use, contact surface 1289 may be directed to a target region 1287 of the sclera. Contact surface 1289 may be angled relative to a longitudinal axis of receiver portion 1288 and/or a needle 1284 of instrument 1282. Due to the angle, eye shield 1280 may be configured to direct needle 1284 to penetrate the sclera at an angle relative to the orthogonal of the sclera. In some embodiments, eye shield 1280 may be configured to direct needle 1284 for nearly tangential penetration of the sclera.

FIG. 12L illustrates another variation of an eye shield. As shown, eye shield 1290 may include a handle portion 1296, where the handle portion 1296 may be configured to be positioned approximately orthogonally relative to a sclera. Eye shield 1290 may further include a retainer portion 1298 configured to retain an instrument 1292 having a needle 1294. Retainer portion 1298 may be configured to pivot relative to handle portion 1296.

Prior to use, retainer portion 1298 may be retracted such that instrument 1292 is parallel or nearly parallel to handle portion 1296. Eye shield 1290 may then be placed against a sclera such that handle portion 1296 is approximately orthogonal to the sclera. Retainer portion 1298 may then be pivoted relative to handle portion 1296 such that needle 1294 is angled relative to the orthogonal of the sclera. In some embodiments, retainer portion 1298 may be pivoted to direct needle 1294 for nearly tangential penetration of the sclera.

FIG. 12M illustrates still another possible geometry of an eye shield. As shown, eye shield 1291 may be generally horseshoe-shaped and configured to accommodate a needle 1293 near a center of eye shield 1291. Similar to eye shield 1260 and eye shield 1270, eye shield 1291 may include a surface 1295 configured to conform to a curvature of an outer portion of a cornea. Eye shield 1291 may therefore guide positioning of needle 1293 laterally of the cornea, using the cornea for alignment.

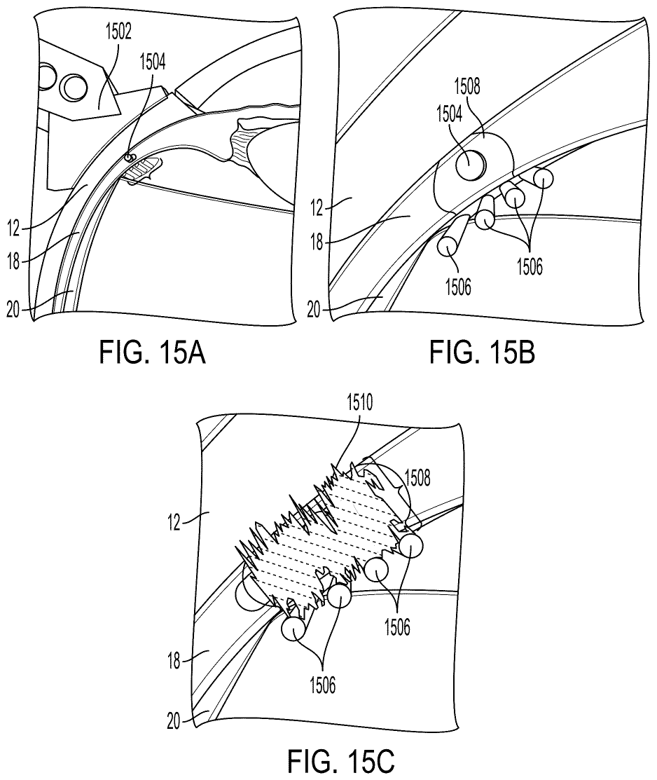

Additional techniques for delivering medicament to regions of an eye and/or treatment ocular conditions and contemplated by this disclosure and may be used in combination with embodiments previously described. FIGS. 15A-15C illustrate a technique for providing gene therapy to an eye. As shown, an instrument 1502 may be used to insert a needle or cannula 1504 and a plurality of electrodes 1506 into an eye. Instrument 1502 may be oriented such that needle or cannula 1504 is positioned beneath a sclera 12 of the eye within a ciliary body 18. Electrodes 1506 may be positioned beneath ciliary body 18 and adjacent to an edge of a retina 20.

A bolus 1508 of medicament may then be delivered via needle or cannula 1504 to ciliary body 18. In some embodiments, the medicament may include DNA plasmids. An electric field 1510 may then be generated between electrodes 1506 and a main body of 1502 and through bolus 1508. Electric field 1510 may result in electroporation, causing cells of ciliary body 18 to become infused with DNA plasmids of bolus 1508. Once infused with DNA plasmids, cells of ciliary body 18 may generate and release gene therapy proteins to other portions of the eye.

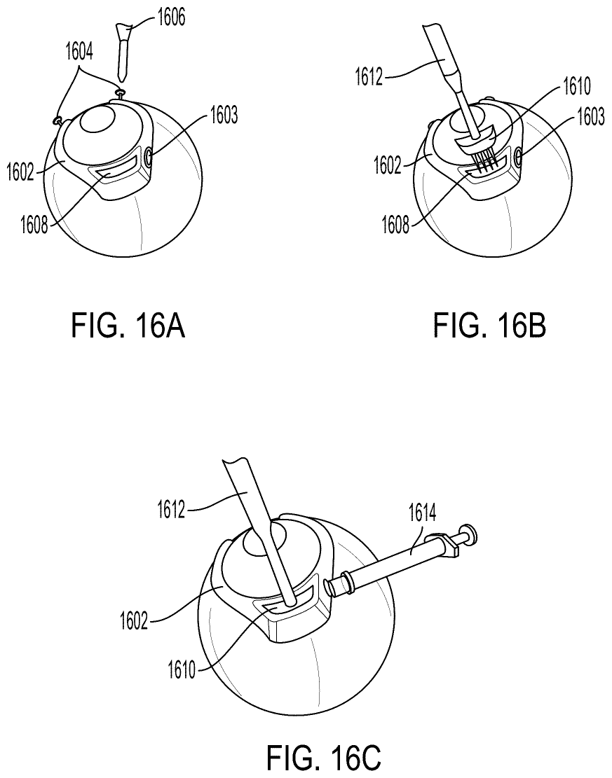

FIGS. 16A-16C illustrate a device configured to execute a technique similar to the technique described with reference to FIGS. 15A-15C. The device may include a ring 1602. Ring 1602 may be secured to a portion of an eye surrounding a cornea by fasteners 1604. In some embodiments, fasteners 1604 may be medical grade pins, trocars, screws, or any other suitable type of fastener. A tool 1606 may be used to insert and/or tighten fasteners 1604. The device may further include a port 1603 and an electrode bay 1608.

As shown in FIG. 16B, an electrode hub 1610 including a plurality of electrodes may be inserted into electrode bay 1608 such that the electrodes penetrate the eye. Electrode hub 1610 may be coupled to a support rod 1612. As shown in FIG. 16C, when electrode hub 1610 is fully inserted within electrode bay 1608, a syringe 1614 may be inserted into port 1603. Port 1603 may be configured to align a needle of syringe 1614 for insertion into a desired portion of the eye adjacent the electrodes. Medicament within syringe 1614 may then be dispensed through the needle and electric field may be generated using the electrodes to cause electroporation, as described previously with reference to FIG. 15C.

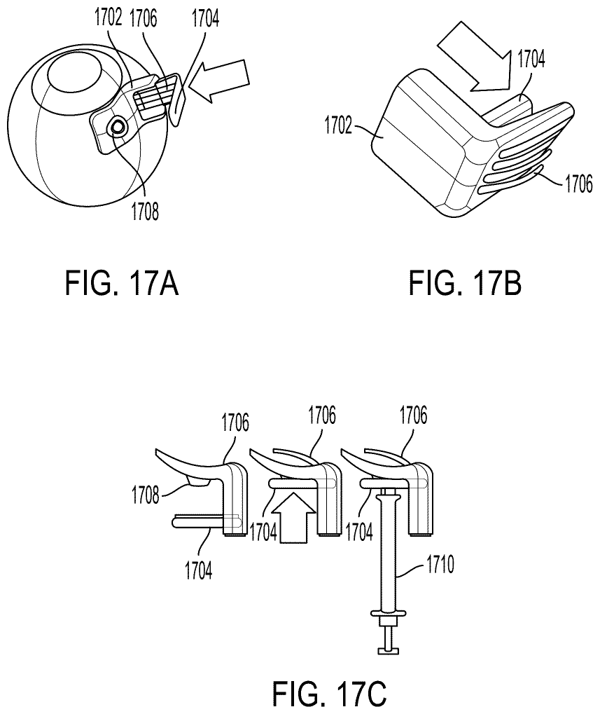

FIGS. 17A-17C illustrate another device configured to execute a technique similar to the technique described with reference to FIGS. 15A-15C. The device may include a receiver 1702. Receiver 1702 may be configured to be placed against a portion of an eye adjacent a cornea. A base of receiver 1702 may be curved to conform to a surface of an eye. The device may further include a port 1708 and an electrode hub 1704 having a plurality of electrodes 1706.

As shown in FIG. 17B, electrode hub 1704 may be advanced into receiver 1702 such that electrodes 1706 extend through receiver 1702. When receiver 1702 is placed on an eye, advancing of electrode hub 1704 may cause plurality of electrodes 1706 to penetrate the eye. As shown in FIG. 17C, when electrode hub 1704 is fully advanced within receiver 1702, a syringe 1710 may be inserted into port 1708. Port 1708 may be configured to align a needle of syringe 1710 for insertion into a desired portion of an eye adjacent the electrodes. Medicament within syringe 1710 may then be dispensed through the needle and an electric field may be generated using electrodes 1706 to cause electroporation, as described previously with reference to FIG. 15C.

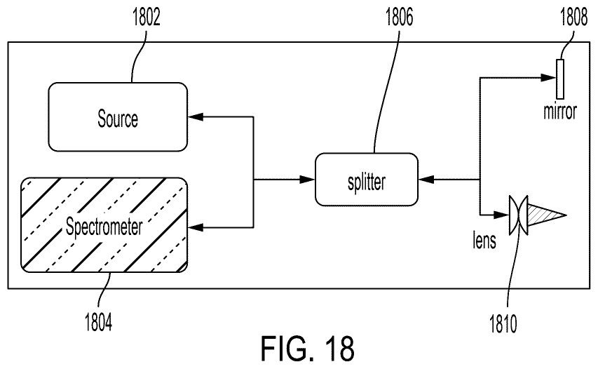

In some embodiments, devices described in this disclosure may be used in conjunction with OCT imaging. FIG. 18 illustrates a block diagram representing a schematic of electronic components of an OCT imaging device. An OCT imaging device may include, for example, a source 1802, a spectrometer 1804, a splitter 1806, a reference mirror 1808, and a lens 1810. Source 1802 may be a light source and, in some embodiments, may be a super-luminescent diode (SLD). Light from source 1802 may be provided to splitter 1806 and subsequently directed to each of reference mirror 1808 and lens 1810. Lens 1810 may be oriented to focus the light from source 1802 on a subject's eye. Light may reflect off each of reference mirror 1808 and the subject's eye, pass through splitter 1806, and arrive at spectrometer 1804 for measurement. Data from spectrometer 1804 may be transmitted to additional circuitry for further analysis.

In some embodiments, source 1802 may include one or more SLDs having a large frequency bandwidth. The frequency bandwidth may be approximately 1300 nm and may allow the OCT imaging device to achieve about 10 μm of axial resolution. A lateral resolution of the OCT imaging device may be about 30 μm, and may depend on one or more of a numerical aperture of lens 1810, power consumption by the OCT imaging device, and signal-to-noise ratio of the OCT imaging device.

In some embodiments, the OCT imaging device may be physically separate from drug delivery devices described herein. For example, the OCT imaging device may be a physically separate device from instrument 100. Data obtained via the OCT imaging device, such as scleral thickness of a subject's eye, may be used to calibrate and/or adjust instrument 100 prior to performing an injection into the subject's eye. In some embodiments, the OCT imaging device may have a handheld form factor. In some embodiments, the OCT imaging device may have a form factor similar to a stylus and may be physically connected to a portable computing device that performs the analysis.

In some embodiments, the OCT imaging device may be integrated with drug delivery devices described herein. For example, the OCT imaging device may be integrated with any of instrument 800, instrument 900, and/or instrument 1100. Data obtained via the OCT imaging device, such as scleral thickness of a subject's eye, may be used to manually calibrate and/or adjust the instrument prior to performing an injection into the subject's eye. In some embodiments, data obtained via the OCT imaging device may be used to automatically calibrate and/or adjust the instrument prior to performing an injection into the subject's eye.

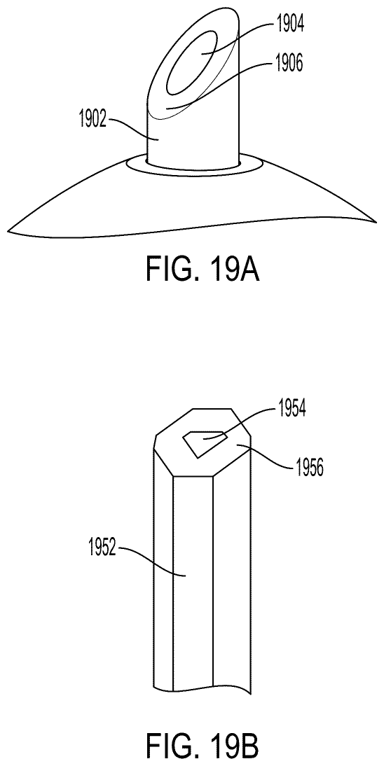

FIGS. 19A and 19B illustrate exemplary needle tips that may be used with drug delivery devices described herein. As shown in FIG. 19A, in some embodiments a drug delivery device may include a conventional stainless steel needle having a distal tip 1902. Distal tip 1902 may have an angled bevel 1906 and a lumen 1904 having a circular or approximately circular cross-section. As shown in FIG. 19B, in some embodiments, a drug delivery device may include a silicon wafer needle 1952. Needle 1952 may have an angled bevel 1956 and a lumen 1954. Use of silicon manufacturing may allow needle 1952 to incorporate a polygonal exterior surface and a polygonal cross-section of lumen 1954. The exterior surface may be octagonal and the cross-section of lumen 1954 may be diamond-shaped in some embodiments. Additionally, use of silicon manufacturing may allow exempt needle 1952 from certain length requirements applicable to stainless steel needles and bevels. Exemption from the length requirements may reduce likelihoods of backflow and inadvertent piercing of a choroid or other eye layers during an injection.

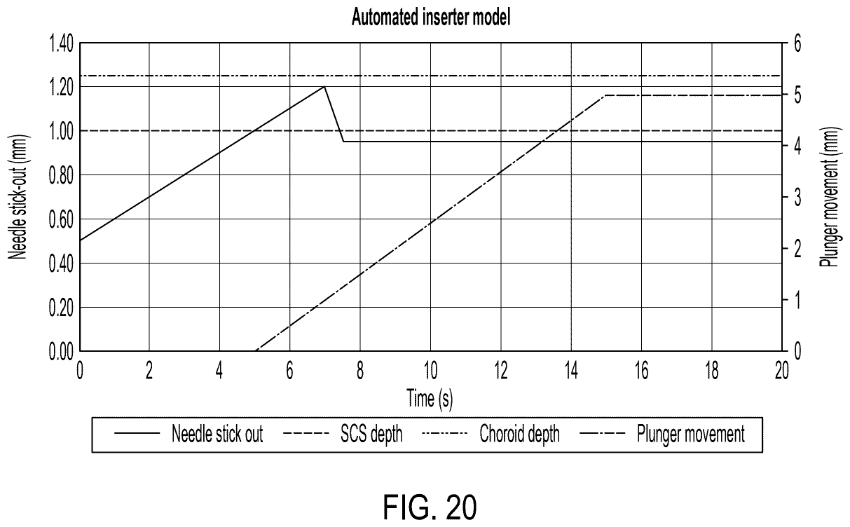

FIG. 20 depicts a graphical model of exemplary operation of drug delivery devices described herein that are configured for automated needle insertion. The model relates needle stick-out, or extension beyond an eye shield, (left vertical axis) and plunger movement distance (right vertical axis) to time (horizontal axis). The model further illustrates SCS depth and choroid depth for comparison. Needle stick-out may represent a distance that needle 610 extends beyond eye shield 608, a distance that needle 810 extends beyond eye shield 808, and/or a distance that needle 1110 extends beyond eye shield 1108, for example. Plunger movement distance may represent a distance plunger 618 moves within cartridge 614 and/or a distance plunger 1118 moves within cartridge 1114.

As shown in FIG. 20, a needle may initially extend about 0.5 mm beyond an eye shield and into a subject's eye when an injection procedure is initiated. The needle may advance at a substantially constant rate for approximately 7 seconds to an extension of about 1.2 mm. The needle may cease advancing before reaching the choroid at a depth of about 1.25 mm. After approximately 5 seconds, the needle may extend to approximate 1 mm, penetrate a sclera, and enter the SCS. As the needle enters the SCS, the plunger may begin to advance at a substantially constant rate and continue for about 10 seconds.

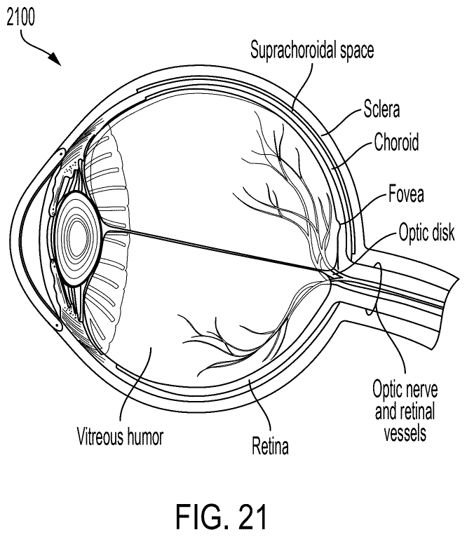

FIG. 21 illustrates an example of a mammalian eye 2100 for reference with the concepts described herein. As shown, the SCS exists between a sclera and choroid. While the present disclosure generally describes delivery of medicament to the SCS, the present disclosure is not so limited. Embodiments of the present disclosure may be used for delivery of medicament to other portions of the eye, such as the fovea, optic disk, optic nerve, retinal vessels, retina, or vitreous humor.

In some embodiments, a drug delivery device may include a needle retraction mechanism configured to retract the needle to an extension of about 0.95 mm. Retraction of the needle may allow drug delivery to continue through a path previously occupied by the needle while reducing a risk of inadvertently piercing the choroid.

An injection using the automated insertion techniques and devices described herein may have a total duration of about 15 seconds. The duration may be adjusted, for example, by altering the rates at which the needle and/or plunger advance.

Listed below are further illustrative embodiments according to the present disclosure:
(1) A device for delivering a medicament to ocular tissue, the device comprising: a housing; a container configured to contain the medicament; a needle in fluid communication with the container, wherein the needle defines a needle axis; a carriage positioned within the housing and coupled to the container and the needle, wherein the carriage is translatable along the needle axis, and wherein the carriage includes a threaded outer surface; and a nut positioned within the housing and engaged with the threaded outer surface, wherein rotation of the nut is configured to cause the carriage and the needle to translate in a distal direction along the needle axis.
(2) The device of item (1), further comprising: an eye shield coupled to the housing at a distal end, wherein the eye shield includes a contoured surface configured to conform to an outer surface of an eye, and wherein the eye shield includes a through-hole positioned about the needle axis, wherein the needle is configured to pass through the through-hole.
(3) The device of item (1), further comprising: an eye shield coupled to the housing at a distal end, wherein the eye shield includes a reference marking configured to align with an anatomical feature of a patient upon placement of the eye shield.
(4) The device of item (1), further comprising: an eye shield coupled to the housing at a distal end, wherein the eye shield includes a reference marking configured to align with an anatomical feature of a patient upon placement of the eye shield, wherein the eye shield is configured to orient the needle for nearly tangential advancement into an eye.
(5) The device of item (1), further comprising: an eye shield coupled to the housing at a distal end, wherein the eye shield is configured to orient the needle for nearly tangential advancement into an eye.
(6) The device of item (1), further comprising: an eye shield coupled to the housing at a distal end, wherein the eye shield includes a contoured surface configured to conform to an outer surface of an eye, wherein a texture of the contoured surface is configured to inhibit slippage of the eye shield relative to the eye.
(7) The device of item (1), further comprising: an eye shield coupled to the housing at a distal end, wherein at least a portion of the eye shield is formed of silicone or rubber.
(8) The device of item (1), wherein the needle is formed of silicon.
(9) The device of item (1), further comprising: a rotary driver configured to apply a torque to the nut during expansion, thereby causing rotation of the nut and translation of the carriage and the needle in the distal direction.
(10) The device of item (1), further comprising: a torsional spring configured to expand from a compressed configuration and apply a torque to the nut during expansion, thereby causing rotation of the nut; a stop configured to move from a first position to a second position, wherein the stop in the first position maintains the torsional spring in the compressed configuration, wherein the stop in the second position allows the torsional spring to expand; and a biasing mechanism configured to urge the stop toward the first position.
(11) The device of item (1), further comprising: a torsional spring configured to expand from a compressed configuration and apply a torque to the nut during expansion, thereby causing rotation of the nut; a stop configured to move from a first position to a second position, wherein the stop in the first position maintains the torsional spring in the compressed configuration, wherein the stop in the second position allows the torsional spring to expand; a biasing mechanism configured to apply force against the stop toward the first position; and a plunger configured to translate through the container along the needle axis, wherein translation of the plunger a predetermined distance causes the biasing mechanism to move the stop from the second position to the first position.
(12) The device of item (1), further comprising: a damper configured to dampen rotation of the nut such that translation of the carriage and the needle occurs at a substantially constant rate.
(13) The device of item (1), an electric motor configured to apply a torque to the nut, thereby causing rotation of the nut.
(14) A device for delivering a medicament to ocular tissue, the device comprising: a housing; a needle defining a needle axis; a carriage positioned within the housing and coupled to the needle, wherein the carriage is translatable along the needle axis, and wherein the carriage includes a threaded outer surface; a nut positioned within the housing and engaged with the threaded outer surface; a motor coupled to the nut; and a controller configured to: receive an activation signal; and cause, in response to the activation signal, the motor to apply a torque to the nut, thereby causing the nut to rotate and causing the carriage and the needle to translate distally along the needle axis.
(15) The device of item (14), wherein the housing further includes an activation button, and wherein the activation signal is indicative of the activation button being depressed.
(16) The device of item (14), further comprising: an eye shield coupled to the housing at a distal end, wherein the eye shield includes a sensor configured to detect contact with the ocular tissue, wherein the housing further includes an activation button and wherein the activation signal is indicative of (1) the activation button being depressed and (2) contact with ocular tissue.
(17) The device of item (14), further comprising: an eye shield coupled to the housing at a distal end, wherein the eye shield includes a sensor configured to detect contact with the ocular tissue, wherein the sensor is a capacitance sensor, a moisture sensor, a temperature sensor, or a combination thereof, wherein the housing further includes an activation button and wherein the activation signal is indicative of (1) the activation button being depressed and (2) contact with ocular tissue.
(18) The device of item (14), further comprising: an eye shield coupled to the housing at a distal end, wherein the eye shield includes a contoured surface configured to conform to an outer surface of an eye, wherein a texture of the contoured surface is configured to inhibit slippage of the eye shield relative to the eye; and wherein at least a portion of the eye shield is formed of silicone or rubber.
(19) The device of item (14), further comprising: a switch; a container configured to contain the medicament; and a plunger configured to translate through the container along the needle axis, wherein translation of the plunger a predetermined distance is configured to toggle the switch; wherein the controller is further configured to: receive a toggle signal indicative of the switch being toggled; and cause, in response to the toggle signal, the motor to cease applying the torque to the nut.
(20) The device of item (14), further comprising: a container configured to contain the medicament; a plunger configured to translate through the container along the needle axis; and a biasing mechanism configured to apply a force to the plunger in a distal direction along the needle axis, wherein a magnitude of the force is such that the plunger translates in the distal direction when a fluid pressure within the container decreases below a predetermined threshold.
(21) The device of item (14), further comprising: a container configured to contain the medicament; a plunger configured to translate through the container along the needle axis; a biasing mechanism configured to apply a force to the plunger in a distal direction along the needle axis; and a controllable retainer configured to selectively release the biasing mechanism; wherein the controller is further configured to: determine that the needle has translated a predetermined distance along the needle axis; and cause, in response to the determination, the controllable retainer to release the biasing mechanism, thereby allowing the biasing mechanism to apply force to the plunger in the distal direction.
(22) The device of item (14), further comprising: a container configured to contain the medicament; a plunger configured to translate through the container along the needle axis; wherein the controller is further configured to: determine that the plunger has translated to a distal end of the container; and cause the nut to rotate in an opposite direction, thereby causing the carriage and the needle to translate proximally along the needle axis.
(23) The device of item (14), further comprising: a light source; wherein the controller is further configured to illuminate the light source to provide a visual indication of medicament delivery progress.
(24) A device for delivering a medicament to ocular tissue, the device comprising: a container configured to enclose the medicament; a needle in fluid communication with the container, wherein the needle defines a needle axis; a collar coupled to the container and the needle, wherein the collar includes a threaded outer surface; an eye shield coupled to a distal end of the device, wherein the eye shield includes a contoured surface configured to conform to an outer surface of an eye, and wherein the eye shield is configured to translate along the needle axis relative to the collar; and a wheel engaged with the threaded outer surface, wherein rotation of the wheel is configured to cause the eye shield to translate along the needle axis.
(25) The device of item (24), wherein at least a portion of the eye shield is formed of silicone or rubber.
(26) The device of item (24), wherein the eye shield includes a through-hole positioned about the needle axis such that the needle protrudes a first distance from the contoured surface.
(27) The device of item (24), wherein the eye shield includes a through-hole positioned about the needle axis such that the needle protrudes a first distance from the contoured surface, and wherein rotation of the wheel causes the first distance to vary.
(28) The device of item (24), wherein the needle is movable between a first position and a second position, wherein in the first position the needle protrudes a first distance from the contoured surface, wherein in the second position the needle protrudes a second distance form the contoured surface, the second distance being greater than the first distance, and wherein rotation of the wheel causes the needle to move from the first position to the second position.
(29) The device of item (24), wherein the wheel is configured to be rotated to predetermined increments.
(30) The device of item (24), wherein the wheel is restrained from translating relative to the eye shield along the needle axis.
(31) A method of delivering a medicament, the method comprising: measuring a thickness of a sclera using optical coherence tomography (OCT), interferometry, ultrasound elastography, or a combination thereof; and delivering the medicament using the device of any one of items (1)-(20).

It will be apparent to those skilled in the art that various modifications and variations can be made in the disclosed devices and methods without departing from the scope of the disclosure. Other aspects of the disclosure will be apparent to those skilled in the art from consideration of the specification and practice of the features disclosed herein. It is intended that the specification and examples be considered as exemplary only.

What is claimed is:

1. A device for delivering a medicament to ocular tissue, the device comprising:
a housing;
a container configured to contain the medicament;
a needle in fluid communication with the container, wherein the needle defines a needle axis;
a carriage positioned within the housing and coupled to the container and the needle, wherein the carriage is translatable along the needle axis, and wherein the carriage includes a threaded outer surface; and
a nut positioned within the housing and engaged with the threaded outer surface, wherein rotation of the nut is configured to cause the carriage and the needle to translate in a distal direction along the needle axis.

2. The device of claim 1, further comprising:
an eye shield coupled to the housing at a distal end, wherein the eye shield includes a contoured surface configured to conform to an outer surface of an eye, and wherein the eye shield includes a through-hole positioned about the needle axis, wherein the needle is configured to pass through the through-hole.

3. The device of claim 1, further comprising:
an eye shield coupled to the housing at a distal end, wherein the eye shield includes a reference marking configured to align with an anatomical feature of a patient upon placement of the eye shield.

4. The device of claim 1, further comprising:
an eye shield coupled to the housing at a distal end, wherein the eye shield includes a reference marking configured to align with an anatomical feature of a patient upon placement of the eye shield, wherein the eye shield is configured to orient the needle for nearly tangential advancement into an eye.

5. The device of claim 1, further comprising:
an eye shield coupled to the housing at a distal end, wherein the eye shield is configured to orient the needle for nearly tangential advancement into an eye.

6. The device of claim 1, further comprising:
an eye shield coupled to the housing at a distal end, wherein the eye shield includes a contoured surface configured to conform to an outer surface of an eye, wherein a texture of the contoured surface is configured to inhibit slippage of the eye shield relative to the eye.

7. The device of claim 1, further comprising:
an eye shield coupled to the housing at a distal end, wherein at least a portion of the eye shield is formed of silicone or rubber.

8. The device of claim 1, wherein the needle is formed of silicon.

9. The device of claim 1, further comprising:
a rotary driver configured to apply a torque to the nut during expansion, thereby causing rotation of the nut and translation of the carriage and the needle in the distal direction.

10. The device of claim 1, further comprising:
a torsional spring configured to expand from a compressed configuration and apply a torque to the nut during expansion, thereby causing rotation of the nut;
a stop configured to move from a first position to a second position, wherein the stop in the first position maintains the torsional spring in the compressed configuration, wherein the stop in the second position allows the torsional spring to expand; and
a biasing mechanism configured to urge the stop toward the first position.

11. The device of claim 1, further comprising:
a torsional spring configured to expand from a compressed configuration and apply a torque to the nut during expansion, thereby causing rotation of the nut;
a stop configured to move from a first position to a second position, wherein the stop in the first position maintains the torsional spring in the compressed configuration, wherein the stop in the second position allows the torsional spring to expand;
a biasing mechanism configured to apply force against the stop toward the first position; and
a plunger configured to translate through the container along the needle axis, wherein translation of the plunger a predetermined distance causes the biasing mechanism to move the stop from the second position to the first position.

12. The device of claim 1, further comprising:
a damper configured to dampen rotation of the nut such that translation of the carriage and the needle occurs at a substantially constant rate.

13. The device of claim 1, an electric motor configured to apply a torque to the nut, thereby causing rotation of the nut.

14. A device for delivering a medicament to ocular tissue, the device comprising:
a housing having;
a needle defining a needle axis;
a carriage positioned within the housing and coupled to the needle, wherein the carriage is translatable along the needle axis, and wherein the carriage includes a threaded outer surface;
a nut positioned within the housing and engaged with the threaded outer surface;
a motor coupled to the nut; and
a controller configured to:
receive an activation signal; and
cause, in response to the activation signal, the motor to apply a torque to the nut, thereby causing the nut to rotate and causing the carriage and the needle to translate distally along the needle axis.

15. The device of claim 14, wherein the housing further includes an activation button, and wherein the activation signal is indicative of the activation button being depressed.

16. The device of claim 14, further comprising:
an eye shield coupled to the housing at a distal end, wherein the eye shield includes a sensor configured to detect contact with the ocular tissue,
wherein the housing further includes an activation button and wherein the activation signal is indicative of (1) the activation button being depressed and (2) contact with ocular tissue.

17. The device of claim 14, further comprising:
an eye shield coupled to the housing at a distal end, wherein the eye shield includes a sensor configured to detect contact with the ocular tissue, wherein the sensor is a capacitance sensor, a moisture sensor, a temperature sensor, or a combination thereof,
wherein the housing further includes an activation button and wherein the activation signal is indicative of (1) the activation button being depressed and (2) contact with ocular tissue.

18. The device of claim 14, further comprising:
an eye shield coupled to the housing at a distal end, wherein the eye shield includes a contoured surface configured to conform to an outer surface of an eye, wherein a texture of the contoured surface is configured to inhibit slippage of the eye shield relative to the eye; and
wherein at least a portion of the eye shield is formed of silicone or rubber.

19. The device of claim 14, further comprising:
a switch;
a container configured to contain the medicament; and
a plunger configured to translate through the container along the needle axis, wherein translation of the plunger a predetermined distance is configured to toggle the switch;
wherein the controller is further configured to:
receive a toggle signal indicative of the switch being toggled; and
cause, in response to the toggle signal, the motor to cease applying the torque to the nut.

20. The device of claim 14, further comprising:
a container configured to contain the medicament;
a plunger configured to translate through the container along the needle axis; and
a biasing mechanism configured to apply a force to the plunger in a distal direction along the needle axis, wherein a magnitude of the force is such that the plunger translates in the distal direction when a fluid pressure within the container decreases below a predetermined threshold.

21. The device of claim 14, further comprising:
a container configured to contain the medicament;
a plunger configured to translate through the container along the needle axis;
a biasing mechanism configured to apply a force to the plunger in a distal direction along the needle axis; and
a controllable retainer configured to selectively release the biasing mechanism;
wherein the controller is further configured to:
determine that the needle has translated a predetermined distance along the needle axis; and
cause, in response to the determination, the controllable retainer to release the biasing mechanism, thereby allowing the biasing mechanism to apply force to the plunger in the distal direction.

22. The device of claim 14, further comprising:
a container configured to contain the medicament;
a plunger configured to translate through the container along the needle axis;
wherein the controller is further configured to:
determine that the plunger has translated to a distal end of the container; and
cause the nut to rotate in an opposite direction, thereby causing the carriage and the needle to translate proximally along the needle axis.

23. The device of claim 14, further comprising:
a light source;
wherein the controller is further configured to illuminate the light source to provide a visual indication of medicament delivery progress.

24. A device for delivering a medicament to ocular tissue, the device comprising:
a container configured to contain the medicament;
a needle in fluid communication with the container, wherein the needle defines a needle axis;
a collar coupled to the container and the needle, wherein the collar includes a threaded outer surface;
an eye shield coupled to a distal end of the device, wherein the eye shield includes a contoured surface configured to conform to an outer surface of an eye, and wherein the eye shield is configured to translate along the needle axis relative to the collar; and
a wheel engaged with the threaded outer surface, wherein rotation of the wheel is configured to cause the eye shield to translate along the needle axis.

25. The device of claim 24, wherein at least a portion of the eye shield is formed of silicone or rubber.

26. The device of claim 24, wherein the eye shield includes a through-hole positioned about the needle axis such that the needle protrudes a first distance from the contoured surface.

27. The device of claim 24, wherein the eye shield includes a through-hole positioned about the needle axis such that the needle protrudes a first distance from the contoured surface, and wherein rotation of the wheel causes the first distance to vary.

28. The device of claim 24, wherein the needle is movable between a first position and a second position, wherein in the first position the needle protrudes a first distance from the contoured surface, wherein in the second position the needle protrudes a second distance form the contoured surface, the second distance being greater than the first distance, and wherein rotation of the wheel causes the needle to move from the first position to the second position.

29. The device of claim 24, wherein the wheel is configured to be rotated to predetermined increments.

30. The device of claim 24, wherein the wheel is restrained from translating relative to the eye shield along the needle axis.

31. A method of delivering a medicament, the method comprising:
measuring a thickness of a sclera using optical coherence tomography (OCT), interferometry, ultrasound elastography, or a combination thereof; and
delivering the medicament using the device of claim 1.
